(19) United States
(12) Patent Application Publication
(10) Pub. No.: US 2007/0055142 A1
(43) Pub. Date: Mar. 8, 2007

(54) Method and apparatus for image guided position tracking during percutaneous procedures

(76) Inventor: William E. Webler, Escondido, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/390,065

(22) Filed: Mar. 14, 2003

(51) Int. Cl. A61B 5/05

(52) U.S. Cl. 600/425

(57) ABSTRACT

Methods and apparatuses for guiding the positioning of a device with a position tracking sensor and pre-recorded images. At least one embodiment of the present invention uses pre-recorded time-dependent images (e.g., anatomical images or diagnostic images) to guide the positioning of a medical instrument (e.g., catheter tips) using real time position tracking during diagnostic and/or therapeutic operations with pre-recorded images. In one embodiment of the present invention, predetermined spatial relations are used to determine the position of a tracked medical instrument relative to the pre-recorded images.

### FIELD OF THE INVENTION

The invention relates to position tracking of medical instruments, and more particularly to image guided position tracking during percutaneous procedures, such as cardiac therapies.

### BACKGROUND OF THE INVENTION

Various embodiments of the present invention will be described and illustrated in the context of cardiac therapies. However, it is understood that present invention is not limited to the position tracking during cardiac therapies.

Cardiovascular diseases account for a large percent of the mortality recently. Many of these deaths are not directly caused by an acute myocardial infraction (AMI). Rather, many patients suffer a general decline in their cardiac function efficiency known as heart failure. In many cases, heart failure is caused by damage accumulated in the heart, such as damage caused by disease, chronic and acute ischemia, and especially as a result of hypertension and Mitral regurgitation. After the diagnosis of the damage in the heart, therapeutic operations can be performed to slow or reverse the progression of heart failure.

FIG. 1 is a schematic drawing of a cross-section of heart 100, which has two independent pumps. One pump includes right atrium 101 and right ventricle 107, which pumps venous blood from an inferior and a superior vena cava to lungs (not shown) to be oxygenated. The other pump includes left atrium 103 and left ventricle 105, which pumps blood from pulmonary veins (not shown) to various body systems, including heart 100 itself. The two ventricles are separated by ventricular septum 121; and, the two atria are separated by the atrial septum (not shown).

The two pumps are activated synchronously in a four-phase operational cycle of a heartbeat. FIGS. 1-3 show diagrams of a heart in different phases of a cardiac cycle.

During a first phase, called systole, right ventricle 107 contracts to eject blood through the pulmonary valve 113 to the lungs, as illustrated in FIG. 1. At the same time, left ventricle 105 contracts to eject blood through aortic valve 115 into aorta 123. Right atrium 101 and left atrium 103 are relaxed during the first phase to begin filling with blood. However, this preliminary filling is limited by the distortion of the atria caused by the contraction of the ventricles.

In the second phase, called rapid filling phase (the start of a diastole), right ventricle 107 relaxes to be filled with blood flowing from right atrium 101 through tricuspid valve 111, which is open during this phase, as illustrated in FIG. 2. Pulmonary valve 113 is closed, so that no blood returns to the right ventricle 107 from the lungs during this phase. Left ventricle 105 also relaxes to be filled with blood flowing from left atrium 103 through mitral valve 117, which is also open during this phase. Similarly, aortic valve 115 is also closed to prevent blood from returning to the left ventricle 105 from the body systems during this phase. The existing venous pressure affects the filling of the two ventricles during this phase. Right atrium 101 and left atrium 103 continue filling during this phase. However, due to relaxation of the ventricles, ventricular pressure is lower than the pressure in the atria, so tricuspid valve 111 and mitral valve 117 stay open and blood flows from the atria into the ventricles.

In the third phase, called diastasis (the last part of the diastole), the ventricles fill very slowly. The slowdown in filling rate is due to the equalization of pressure between the venous pressure and the intra-cardiac pressure. In addition, the pressure gradient between the atria and the ventricles is also reduced.

In the fourth phase, called atrial systole (the end of the diastole and the start of the systole of the atria), the atria contract to force additional blood into the ventricles, illustrated in FIG. 3. Although there are no valves guarding the veins entering the atria, there are some mechanisms to inhibit backflow during atrial systole. In left atrium 103, sleeves of atrial muscle extend for one or two centimeters along the pulmonary veins and tend to exert a sphincter-like effect on the veins. In right atrium 101, a crescentic valve forms a rudimentary valve called the eustachian valve which covers the inferior vena cave. In addition, there may be muscular bands which surround the vena cava veins at their entrance to right atria 101.

Although the heart is full of blood, it cannot receive oxygen and nutrients from the blood inside the ventricles and atria. The heart muscle must rely on the arteries on the surface of the heart, known as the coronary arteries, to nourish it and keep it working properly. There are three main coronary arteries: the right coronary artery, the left anterior descending coronary artery and the circumflex coronary artery. These three arteries branch into thousands of small arteries like a tree trunk branches into limbs, bringing oxygen and nutrients to the heart muscle cells.

Coronary artery disease is the narrowing or obstruction of the blood vessels that supply blood and oxygen to the heart muscle, caused by fatty deposits on the walls of the arteries. These fatty deposits gradually build up, causing a marked reduction of blood flow and thus, oxygen and nutrients to the heart. The lack of blood flow (primarily oxygen deprivation) to the heart muscle can cause damage to the heart, resulting ischemia and myocardial infraction. Thus, If the blood flow is significantly reduced, some form of medical treatment becomes necessary.

One of the most common non-surgical treatments for opening obstructed coronary arteries is Percutaneous Transluminal Coronary Angioplasty (PTCA), in which a catheter is inserted into a blood vessel under the skin to reach and reshape the coronary artery. Typically, x-ray is used to guide the advance of the angioplasty catheter (balloon-tipped) along the blood vessel to the heart in a procedure known as cardiac catheterization.

During cardiac catheterization, a physician inserts a long, thin tube into a blood vessel in the groin or arm of a patient. The tube is gently directed to the heart and to the origin of the coronary arteries. Contrast or Dye is then injected into the coronary artery while x-ray pictures are taken. The dye in the coronary arteries is seen by the x-ray as a dark line. A disruption of the dark line may signify an area of plaque build-up inside the wall of the artery. In another example, dye can be injected into the pumps of the heart in order to see how well the heart muscle is contracting and how well the valves are working. Pressure measurements are also typically performed during cardiac catheterization using a pressure sensor connected to the proximal end of a catheter lumen or mounted on the tip of the catheter.

Catheters can also be used to map the geometry of the heart and time related changes in the geometry of the heart (e.g., using the NOGA system from Biosense Webster, Inc.). FIG. 4 shows a prior art method of mapping the geometry of the heart (see U.S. Pat. No. 6,285,898 for more details). In FIG. 4, distal tip 141 of mapping catheter 131 is inserted into heart 100 and brought into contact with heart 100 at a location (e.g., 133 or 135). The position and orientation of tip 141 is determined using position sensor 137 (e.g., a sensor as described in U.S. Pat. No. 5,391,119 or in U.S. Pat. No. 5,443,489), which typically requires an external magnetic field generator (not shown) to determine the position and orientation of the tip. Alternatively, other position sensors as known in the art can be used, for example, ultrasonic, RF and rotating magnetic field sensors. Alternatively or additionally, tip 141 is marked with a marker whose position can be determined from outside of the heart, for example, a radio-opaque marker for use with a fluoroscope. At least one reference catheter can be inserted into the heart and placed in a fixed position relative to the heart so that, by comparing the positions of mapping catheter 131 and the reference catheter, the position of tip 141 relative to the heart can be accurately determined even if heart 100 exhibits overall motion within the chest. The positions can be compared at least once every cardiac cycle, more preferably, during diastole. Alternatively, position sensor 137 determines the position of tip 141 relative to the reference catheter, for example, using ultrasound, so no external sensor or generator is required.

For example, U.S. Pat. No. 6,216,027 describes a system for electrode localization using ultrasound, in which one or more ultrasound reference catheters are used to establish a fixed three-dimensional coordinate system within a patient's heart using principles of triangulation. The coordinate system is represented graphically in three-dimensions on a video monitor to aid the clinician in guiding other medical devices, which are provided with ultrasound transducers, through the body to locations at which they are needed to perform clinical procedures.

After determining multiple locations of the tip of the mapping catheter, brought in contact with different locations on a surface of the heart, a surface can be reconstructed from the data points.

Each position value for the tip of the mapping catheter has an associated time value, preferably relative to a predetermined point in the cardiac cycle. Multiple position determinations are performed, at different points in the cardiac cycle, for each placement of the tip. Thus, a geometric map comprises a plurality of geometric snapshots of the heart, each snapshot associated with a different instant of the cardiac cycle. The cardiac cycle is preferably determined using a standard Electrocardiogram (ECG, sometimes abbreviated as EKG) device. Alternatively or additionally, a local reference activation time is determined using an electrode on the catheter.

Electrocardiogram (ECG) is a non-invasive test that records the electrical activity generated by the heart to yield information about the heart rhythm and rate, presence of an old or ongoing heart attack (myocardial infarction), or evidence of impaired blood supply (ischemia).

When heart rate varies, but is not arrhythmic, the interval between each heartbeat is treated as one time unit. When the heart rate varies, either naturally, or by choice (manual pacing), position and other sensed values are binned according to electrocardiogram (ECG) or electrocardiogram morphology (i.e. time after “R” wave), beat length, activation location, relative activation time or other determined cardiac parameters. Thus, a plurality of maps may be constructed, each of which corresponds to one bin.

However, such a system for mapping a heart is time consuming, difficult to use and very limited in resolution and quality in the images it can produce for the purpose of guiding a cardiac therapy.

### SUMMARY OF THE DESCRIPTION

Methods and apparatuses for position tracking guided with pre-recorded images are described here. Some embodiments of the present inventions are summarized in this section.

At least one embodiment of the present invention uses pre-recorded time-dependent images (e.g., anatomical images or diagnostic images) to guide real time position tracking of medical instruments (e.g., catheter tips) during diagnostic and/or therapeutic operations. In one embodiment of the present invention, predetermined dimensional relations are used to determine the position of a tracked medical instrument relative to the details depicted in the pre-recorded images.

In one embodiment of the present invention, a method of displaying images of a heart includes: storing a time-related sequence of cardiac images which are associated with at least one cardiac data parameter (e.g., Electrocardiogram (ECG), heart sound, blood pressure, ventricular volume, pulse wave, heart motion, and cardiac output); determining a position of a portion of a medical instrument relative to the heart; determining at least one measurement of the at least one cardiac data parameter; selecting at least one cardiac image from the time-related sequence of cardiac images according to the at least one measurement of the at least one cardiac data parameter; and overlaying a representation of the portion onto the at least one cardiac image to indicate its position relative to the heart. In one example, the at least one cardiac image is displayed to show the portion of the medical instrument in relation to the heart in real time; and, the at least one measurement is determined substantially contemporaneously with the determining of the position. In one example, the time-related sequence of cardiac images is correlated with measurements of the at least one cardiac data parameter; each of the time-related sequence of cardiac images comprises a pixel image; and, the time-related sequence of cardiac images are generated from an imaging system based on at least one of: a) Magnetic Resonance Imaging; b) X-ray imaging; and c) ultrasound imaging. In one example, the at least one cardiac image is selected based on a hemodynamic parameter or other physiologic parameter (e.g., blood pressure, heart rate, ECG, respiration rate, respiration cycle, hydration state, blood volume, and sedation state) determined substantially contemporaneously with the determining of the position.

In one embodiment of the present invention, a method of displaying images of a heart includes: determining a first state of an organ from at least one first measurement of at least one parameter; and determining a first image from a plurality of images of the organ to display the organ in the first state, where the plurality of images correspond to the organ in a plurality of states. In one example, a first position of a portion of a medical instrument is determined relative to the organ in the medical operation when the organ is in the first state; and, the first image is displayed with a representation of the portion of the medical instrument overlaid on the first image according to the first position. The first image is displayed substantially in real time to show the portion of the medical instrument in relation with the organ. In one example, to determine the first position, position information of the portion of the medical instrument is received from a position determination system when the organ is in the first state, where the first position is determined from the position information from aligning both a first coordinate space of the position determination system and a second coordinate space of the plurality of images with respect to the organ; the first coordinate space and the second coordinate space are aligned with respect to the organ using a transformation to align the first coordinate space and the second coordinate space with respect to a reference object; the reference object is a platform supporting a host of the organ; and, the host has a fixed position relative to the platform both when the plurality of images are generated in an imaging system and when the position information is determined in the position determination system.

In one embodiment of the present invention, a method of displaying images of a heart or other organ includes: storing a plurality of images of an organ which is associated with at least one parameter; and automatically playing back the plurality of images in real time according to real time measurements of the at least one parameter. In one example, the position information of a portion of a medical instrument is received in real time during the medical operation; and, a representation of the portion of the medical instrument is overlain on displayed ones of the plurality of images to illustrate a position of the portion of the medical instrument in relation with the organ according to the position information. In one example, a position of the portion of the medical instrument is determined relative to the organ in a displayed one of the plurality of images from the position information; and, the position information is determined by a real time position tracking system based on one of: a) magnetic field; b) ultrasound; c) radio frequency signal; and d) light. In one embodiment of the present invention, the plurality of images are obtained (e.g., using a Magnetic Resonance Imaging (MRI) system, or a Computer Tomography (CT) system) before said playing back.

In one embodiment of the present invention, a platform is used to support and transport a patient between known locations in an imaging system and a position determination system. The position of the organ/body relative to the platform is held relatively constant so that the person/patient/animal/object is in a single relatively fixed position relative to the platform both during imaging and during device position sensing. The platform is used as a reference object in overlaying a representation of the position determined by the position determination system on the image obtained from the imaging system. For example, the location of the platform in the imaging system is known in the image coordinate system (e.g., the platform is at a position determined in real-time, or at a predetermined position, in the imaging system); and, after the transport of the platform from the imaging system to the position determination system, the location of the platform is similarly known in the coordinate system of the position determination system. Additionally, the units (e.g., inches, millimeters, radians, degrees, etc.) of the imaging coordinate system and the position determination system are known. Thus, a transform is generated to align (to the same scale, orientation and origin) the coordinate systems of the imaging system and the position determination system such that a real-time representation of the portion of the medical device with the position sensor/transducer (or sensors/transducers) can be overlaid on the recorded organ image(s) in the same coordinate system relative to the platform. Such an alignment may be most easily performed/calibrated using an appropriate imaging/positioning phantom(s) that is (are) attached to the platform prior to any procedure (e.g., at regular maintenance intervals).

In one aspect, a method to display an image for guiding a medical operation includes: collecting an image of an organ of a person, where the image is generated by an imaging system and in a coordinate system of the imaging system while the person is in a first position relative to a platform in the imaging system; collecting first position information that represents a position of a portion of a medical instrument in a coordinate system of a position determination system, where the first position information is generated by the position determination system while the person is in the same first position relative to the platform in the position determination system after the person and the platform are transported from the imaging system to the position determination system; determining a second position that is the position of the portion of the medical instrument relative to the organ depicted in the image and is derived from the first position information; and, overlaying a representation of the portion of the medical instrument onto the image of the organ according to the second position to display the position of the portion of the medical instrument relative to the organ. In one example, the second position is derived using predetermined data (e.g., platform position data, coordinate transform, or others) that relates the coordinate system of the position determination system and the coordinate system of the imaging system; the predetermined data specifies a transformation to align a position of the platform, which is generated by the position determination system when the platform is in a third position that is in the position determination system, with a corresponding position of the platform on an image, which is generated by the imaging system when the platform is in a fourth position that is in the imaging system; the predetermined data includes data representing a position and orientation of the platform in the coordinate system of the position determination system when the platform is in the third position; and, the predetermined data further includes data representing a position and orientation of the platform in the coordinate system of the imaging system when the platform is in the fourth position. In one example, the image of the organ is collected when the platform is in the fourth position; and, the first position information is collected when the platform is in the third position. In another example, second position information is collected for aligning the coordinate systems of the position determination system and the imaging system, where the second position information represents a position of the platform relative to the third position when the first position information is collected. In a further example, third position information is collected for aligning coordinate systems of the position determination system and the imaging system, where the third position information represents a position of the platform relative to the fourth position when the image of the organ is collected. In one example, the predetermined data is collected before the image of the organ is collected (e.g., at a maintenance interval without the person on the platform).

In another aspect, a method to determine a position of a portion of a medical instrument relative to an organ (e.g., a heart) includes: receiving data for aligning positions determined by a position determination system relative to a reference object with corresponding locations on images generated from an imaging system relative to the reference object (e.g., a platform for supporting the host of the organ, phantoms attached to the platform, the organ itself, an object or objects attached to the host, marks or markers on the host or organ, an object or objects in or on the organ), where the reference object is at a first position in the imaging system when the images are generated, and where the reference object is at a second position in the position determination system when the positions are determined; receiving position information of the portion of the medical instrument determined by the position determination system (e.g., relative to the position determination system or relative to the reference object), where the position information is determined when the reference object is in a third position relative to the organ in the position determination system; and, determining a position of the portion of the medical instrument relative to the organ depicted in a first image from the received position information and the received data for aligning, where the first image is generated by the imaging system when the reference object is in the same third position relative to the organ in the imaging system. In one example, the received data for aligning comprises at least one of: a) data representing a position of the reference object determined by the position determination system when the reference object is in the second position; b) data representing an orientation of the reference object determined by the position determination system when the reference object is in the second position; c) data representing a position of the reference object in an image generated from the imaging system when the reference object is in the first position; and, d) data representing an orientation of the reference object in an image generated from the imaging system when the reference object is in the first position. In one example, the position of the portion of the medical instrument relative to the organ is determined using: a) data indicating a position of the reference object relative to the second position when the position information is determined; and/or b) data indicating a position of the reference object relative to the first position when the first image is generated. In one example, the first image is selected from a plurality of images of the organ according to at least one measurement of at least one parameter related to the organ; the at least one measurement is generated substantially contemporaneous with a time at which the position information is determined; and, the plurality of images is associated with different measurements of the at least one parameter.

The present invention includes methods and apparatuses which perform these methods, including data processing systems which perform these methods, and computer readable media which when executed on data processing systems cause the systems to perform these methods.

Other features of the present invention will be apparent from the accompanying drawings and from the detailed description which follows.

### BRIEF DESCRIPTION OF THE DRAWINGS

The present invention is illustrated by way of example and not limitation in the figures of the accompanying drawings in which like references indicate similar elements.

FIGS. 1-3 show diagrams of a heart in different phases of a cardiac cycle.

FIG. 4 shows a prior art method of mapping the geometry of the heart.

FIG. 5 shows a method to guide a cardiac therapy using a diagnostic image according to one embodiment of the present invention.

FIG. 6 illustrates a diagram of one embodiment of a catheter assembly.

FIG. 7 illustrates a diagram of one embodiment of the first catheter of FIG. 6.

FIG. 8 illustrates a cross-section of the stiff portion of the first catheter shown in FIG. 7.

FIG. 9 illustrates a cross-section of the flexible portion of the first catheter shown in FIG. 7.

FIG. 10 illustrates a diagram of one embodiment of the second catheter of FIG. 6.

FIG. 11 illustrates a cross-section of the stiff portion of the second catheter of FIG. 10.

FIG. 12 illustrates a cross-section of the deflectable portion of the second catheter of FIG. 10.

FIG. 13 illustrates a diagram of the third catheter of in FIG. 6.

FIG. 14 illustrates a cross-section of the third catheter of FIG. 13.

FIG. 15 illustrates various methods to prepare images for guiding real time position tracking according to embodiments of the present invention.

FIGS. 16-17 illustrate a method to align coordinates of a position tracking system with coordinates of an imaging system according to one embodiment of the present invention.

FIG. 18 illustrates alternative methods to register coordinates of a position tracking system with coordinates of an imaging system according to embodiments of the present invention.

FIG. 19 illustrates a method to map real time tracked positions to corresponding pre-recorded images according to one embodiment of the present invention.

FIG. 20 illustrates another method to generate simulated real time cardiac images from pre-recorded images and real time measurements of cardiac parameters according to one embodiment of the present invention.

FIG. 21 shows a block diagram example of a data processing system which may be used with the present invention.

FIG. 22 shows a flow chart for a method to determine an image from a plurality of pre-recorded images to guide real time position tracking during a percutaneous procedure according to one embodiment of the present invention.

FIG. 23 shows a flow chart for a method of image guided real time device positioning using real time position tracking for a cardiac therapy according to one embodiment of the present invention.

FIG. 24 shows a flow chart for a method to superpose a position determined by a position tracking system on an image from an imaging system according to one embodiment of the present invention.

FIG. 25 shows a flow chart for a detailed method to superpose a position determined by a position tracking system on an image from an imaging system according to one embodiment of the present invention.

FIG. 26 shows a flow chart for a detailed method to guide a cardiac therapy using pre-recorded cardiac images according to one embodiment of the present invention.

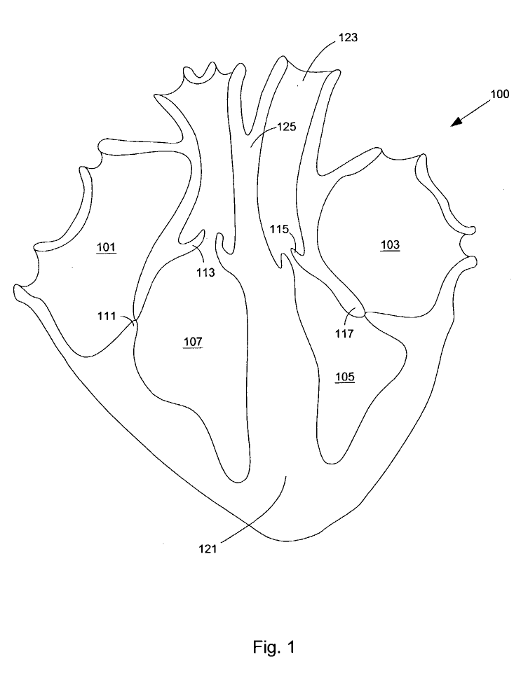

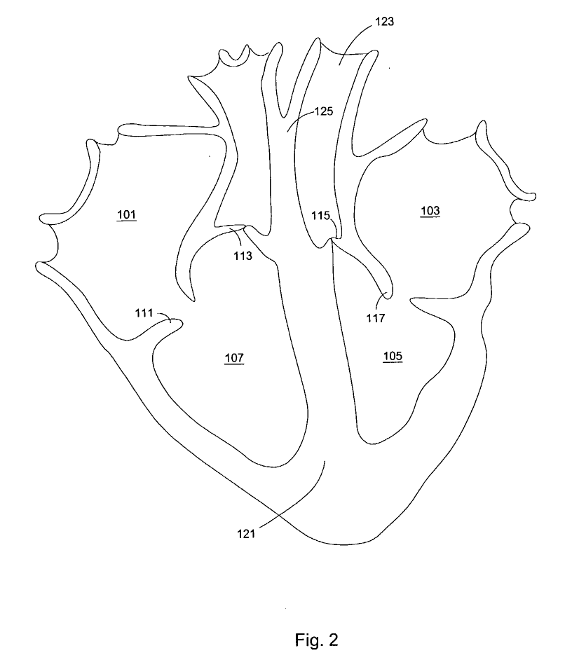

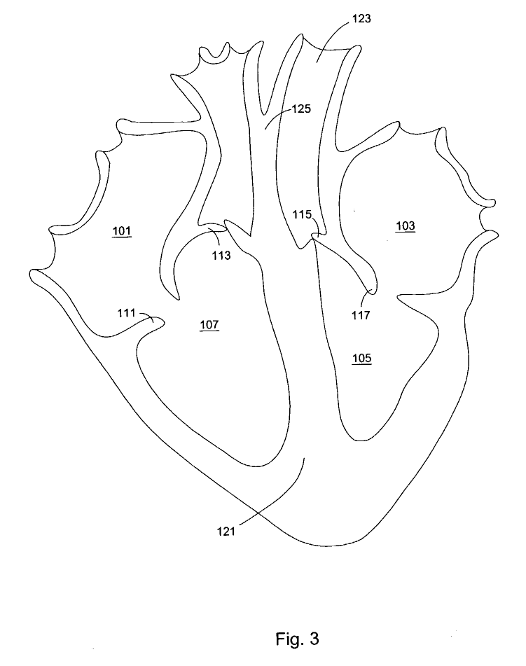

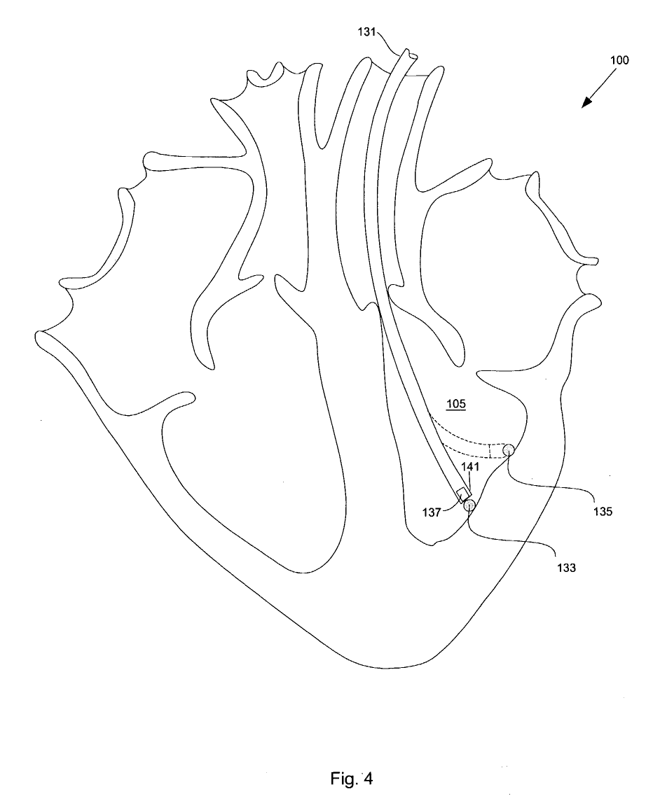

### DETAILED DESCRIPTION

The following description and drawings are illustrative of the invention and are not to be construed as limiting the invention. Numerous specific details are described to provide a thorough understanding of the present invention. However, in certain instances, well known or conventional details are not described in order to avoid obscuring the description of the present invention.

At least one embodiment of the present invention seeks to use pre-recorded time-dependent images (e.g., anatomical images or diagnostic images) to guide real time position tracking of medical instruments (e.g., catheter tips) during diagnostic and/or therapeutic operations. Although examples of embodiments of the present inventions are illustrated using cardiac therapies (e.g., cell therapy, scaffolding, angiogenesis, and others), it will be apparent to one skilled in the art from this description that similar approaches can also be used in other diagnostic and/or therapeutic operations.

In a cardiac therapy, a catheter can be used to reach the heart and apply therapy to the diagnostically relevant areas. Further, the therapy may be applied at a required spacing (e.g., to control dose level at proper spots).

A NOGA system is currently available from Biosense Webster, Inc., for electromechanical mapping of a heart. The NOGA system maps a heart based on a magnetic catheter tip location/position and orientation determination system, as described in the background section. By ensuring that the catheter tip is in contact with the ventricular surface when a location is recorded, a map of the intra-ventricular surface can be created. However, to construct an image of the intra-ventricular surface of a heart using the NOGA system, a physician must gather enough points by positioning the mapping catheter tip at various locations of the intra-ventricular surface, which is a time consuming operation.

Further, since the NOGA system relies on the joining of discrete location points to build an image, the image quality is very poor; and, it can only create a surface or line image of the locations that the catheter has been positioned. In theory, the 3-D location/position determination system could also be used to create a line map of a vascular bed. However, this would be even more time consuming and, therefore, is impractical compared to the currently used fluoroscopy/angiographic procedures. To map the vascular bed, the physician would have to slowly discover, traverse and record every vessel branch with the catheter; and, the amount of time and difficulty that would be involved makes it impractical.

Thus, in a very real sense, the current use of a 3-D location/position (and in some cases, also orientation) determination system is not effective in guiding a cardiac therapy. It simply records and displays the places that the physician has positioned the catheter tip. The physician cannot simply use the 3-D location/position determination system to guide the catheter/device to the locations requiring therapy. Instead, the physician has to systematically move the catheter tip to all the locations where therapy might be required, using the physician's anatomical knowledge and the limited diagnostic tools available during the procedure. In the process, the catheter tip goes to many locations that do not need therapy. Thus, a currently available conventional system is time consuming, difficult to use and very limited in the images it can produce and the therapies it can assist.

According to one embodiment of the present invention, after a patient sees their physician with a cardiac aliment, the physician diagnoses a disease for which the treatment requires 3-D anatomical and diagnostic information to guide the application of the therapy. The patient is imaged using a 3-D imaging system and the image matrices are recorded. The physician (or, a specialist(s) and/or a technician) examines the images to confirm the diagnosis, annotate/color/outline the tissues, sites or surfaces of interest, select special views, add other diagnostic information, etc. The recorded images are loaded into the 3-D location system in the Cath Lab. The therapeutic (when desired, with some complimentary diagnostic capabilities) or delivery catheter is inserted into the patient using normal procedures, devices and equipments. A calibration operation is performed to time (synchronize), align, orient and scale the 3-D location system's location data and that of the recorded images with the patient's current ECG and anatomy (also with their breathing, if a part of the image data). The physician positions the catheter to a previously diagnosed position for therapy, guided by the images shown on his monitor. The monitor will show the selected image view and the catheter's location relative to that image in real time. Alternatively, the physician may guide the catheter to a position, previously diagnosed as suspected of requiring therapy using the image on the monitor. Then in conjunction with the diagnostic information from the catheter, the physician decides if therapy should be administered at that location. In another alternative, the physician will position a delivery catheter and/or an implant to the desired location, guided by the images shown on the monitor. If desired, any additional diagnostic data and/or the actual therapeutic location is recorded and annotated on the display.

According to embodiments of the present invention, a real time 3-D catheter location determination system is used with recorded 3-D anatomical/diagnostic images to guide an operation in order to accurately position the therapy and/or a therapeutic device within the anatomy.

For example, Nuclear Magnetic Resonance (NMR) and X-ray based 3-D imaging systems, such as Magnetic Resonance Imaging (MRI), Magnetic Resonance Angiography (MRA), XMRI, Multi-axis Fluoroscopy, Computed Tomography (CT) and Electron Bean Computed Tomography (EBCT) can be used to produce 3-D cardiac images. Such images have been used to provide diagnostic information. However, at present, it is very difficult, if not impossible, to guide a therapy in a 3-D space using the real time images that many of these systems can produce.

For example, a cardiac MRI is a non-invasive test that uses magnetic fields, transmitted radio frequency waves and the differing magnetic properties of a body to obtain high-resolution pictures of the heart and surrounding structures. It also permits assessment of heart valves and overall heart function. Cardiologists use cardiac MRIs generally to evaluate for the presence of underlying heart disease. More specific uses include evaluating the right ventricle (the right pumping chamber) when an arrhythmia is suspected of arising from there (the right ventricle is difficult to evaluate using other techniques), and ascertaining the origin and course of the coronary arteries when there is suspicion abnormal conditions. Certain individuals are born with abnormally coursing arteries that predispose them to arrhythmias.

However, magnetic resonance based imaging systems are not widely available in the therapeutic setting (i.e. Catheter Laboratory), because they are expensive and susceptible to electromagnetic interference, requiring special Radio Frequency Interference (RFI) shielding and excluding the use of magnetically susceptible materials in their vicinity. Therefore, magnetic resonance systems are slow to be adopted in therapeutic setting; and, the use of magnetic resonance systems may exclude certain patient populations from the treatment (e.g., because of susceptible pacemakers or other implants) or exclude certain devices/materials from being used in the therapy.

X-ray based imaging systems may expose the operator and the patient to unacceptably high long-term doses of radiation in real time guiding therapy operations, especially when guidance is required often and/or for an extended period of time (e.g., more than a few seconds). The risk of x-ray exposure is the primary impetus for the introduction of MRI to the Catheter Laboratory (Cath Lab), even though MRI compatible devices and MRI real time imaging and guidance of devices are still in their infancy. The current cutting edge Cath Lab MRI systems are XMRI systems. That is, the MRI system is paired with a fluoroscope (the X is for X-ray), so that when the MRI images are not adequate, the patient can be quickly and easily imaged with the fluoroscope in the conventional manner.

According to embodiments of the present invention, images from imaging systems (such as the X-ray based imaging system, the magnetic resonance based imaging systems, ultrasound based imaging systems, or others) are recorded and gated (or correlated) with measurements of cardiac parameters so that the images can be played back in sequence according to the real time cardiac parameters to produce the illusion of real time 3-D cardiac images. For example, images can be stored (and averaged when desired) based on their collection time after the ECG “R” wave (preferred) (or other ECG features, or other cardiac cycle indicators, such as pressure waveforms, valve noises, etc.) so that their display can be synchronized with the real time ECG “R” wave (or other measurements) to produce the illusion of real time 3-D cardiac images. Thus, the pre-recorded images from an imaging system can be played back according to the real time measurements to replace the fluoroscope for guiding the cardiac procedure.

These recorded images have the properties needed to guide any real time cardiac therapy. However, these recorded images are not taken at real time so that they do not show the real time location/position of a therapeutic catheter (or other device) in relation with the heart. To guide the therapeutic operation, the image corresponding to the real time state of the heart is selected from the recorded images according to the real time measurements of cardiac parameters (and other parameters, such as chest movement, etc). A position determination (or tracking) system (e.g., sonic, magnetic, or radio frequency based 3-D location and orientation determination systems) can provide the real time catheter position data with little risk to the operator/patient (in contrast to x-ray systems) and with few material and location limitations (in contrast to nuclear magnetic resonance systems). In one embodiment of the present invention, a catheter position determined by the position tracking system is overlaid on the displayed image, selected from the pre-recorded images at real time according to the real time cardiac parameters, to guide a therapeutic operation.

In one embodiment of the present invention, the patient is required to be relatively hemodynamically stable (e.g., no rapid changes in heart rate/blood pressure) so that the pre-recorded images of the heart accurately represent the state of the heart in real time playback, when synchronized to the real time cardiac parameters. If the patient is stable hemodynamically and physically during the image data collection, the degree of image contrast and the detail in the recorded images are very high, when compared with the real time images produced by the currently available modalities.

Further, the recorded images of nuclear magnetic resonance or x-ray based 3-D imaging systems can also be modified to enhance, color, and/or outline structures/regions of interest and/or to indicate the diagnostic state of a structure or tissue, as determined by the imaging modality or another diagnostic modality. These images can also be recorded in conjunction with a contrast media injection to help identify the outlines of a vascular bed or cardiac chamber(s). The recorded image matrices can also be modified to show different views, image slices, surfaces and the like from a collection of image matrices.

For example, contrast-enhanced MRI can be used to identify reversible myocardial dysfunction. After the administration of contrast material, contrast-enhanced MRI based on the different normal wash-in and wash-out rates demonstrated by healthy and non-healthy myocardium can be used to evaluate the myocardium for viability (Infarct vs. Ischemic). Delayed enhancement imaging suppresses signal from normal myocardium while demonstrating increased signal in infarcted areas of the myocardium where pooling of contrast agents (e.g., gadolinium) occurs to generate high-resolution images, which can offer important diagnostic information to a trained physician when the presence, age and extent of a myocardial infarct is in question. Examples of such delayed enhancement imaging, using CT or MRI based imaging systems, can be found in: Circulation, Vol. 106, No. 9, 1083-1089, 2002; Circulation, Vol. 106, No. 8, 957-961, 2002; Circulation, Vol. 106, No. 2, discussion e6, 2002; Circulation, Vol. 104, No. 9, 1083, 2001; The New England Journal of Medicine, Vol. 343, No. 20, 1445-1453, 2000; Circulation, Vol. 99, No. 15, 2058-2059, 1999. Currently, a spiral multi-slice CT or EBCT imaging system can produce high resolution diagnostic images with delayed enhancement. MR based imaging systems are typically noisier than a CT based imaging system. Thus, some MR based imaging systems bin the signals based on ECG signals to obtain averaged images with a higher signal to noise ratio.

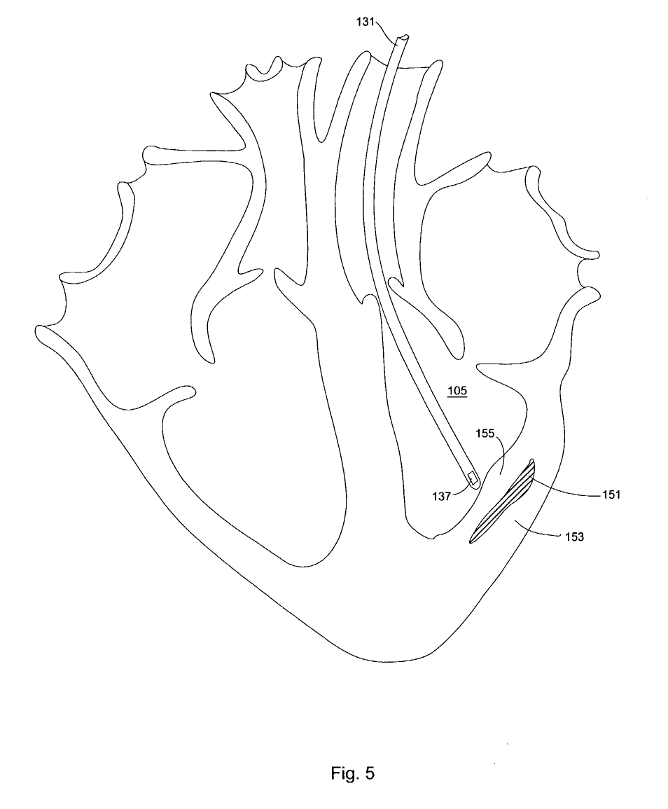

FIG. 5 shows a method to guide a cardiac therapy using a diagnostic image according to one embodiment of the present invention. After a diagnostic image is recorded and analyzed to identify the ischemic region, the diagnostic image is used to guide the treatment so that the treatment can be applied precisely at the desirable locations and spacing (e.g., to apply doses at proper spacing, to avoid injecting doses at a same spot, to apply doses only at diseased regions). For example, ischemic region 151 may be inside the wall, hidden between healthy tissues 153 and 155. When a representation of catheter tip 137 is superposed on the diagnostic image at real time to shown the position of the catheter tip relative to the dysfunctional region, a physician can precisely target the treatment. Details of overlaying a representation of the catheter tip on a diagnostic image according to real time conditions will be described below. It is noticed that it would be very difficult to identify ischemic region 151 when a cardiac mapping system based on joining discrete points is used, since ischemic region 151 is not at the surface of the heart. As discussed above, it is difficult and time consuming to guide a therapy using a reconstructed image based on discrete points contacted by the mapping catheter tip.

Although various catheters known to the person skilled in the art can be used with the present invention for image guided operations, a detailed example of a catheter assembly for image guided operations according to one embodiment of the present invention is described below.

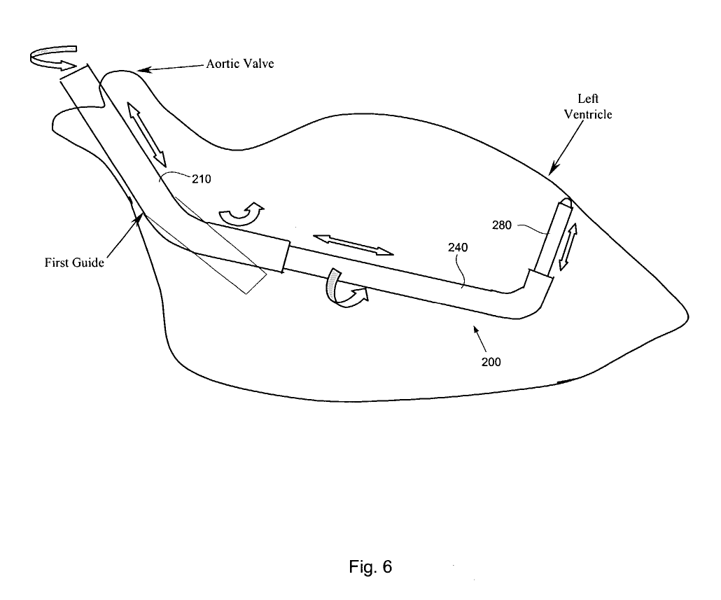

FIG. 6 illustrates a diagram of one embodiment of a catheter assembly 200. The catheter assembly 200 is shown to be extending from the aortic valve into the left ventricle of the heart. Catheter assembly 200 includes a first catheter 210, a second catheter 240, and a third catheter 280. The second catheter 240 fits coaxially into the first catheter 210. The third catheter 280 fits coaxially in the second catheter 240. Each catheter is free to move longitudinally and rotationally relative to the other catheters. In one embodiment, the first catheter 210 may be an outer guide. In one embodiment, the third catheter 280 may be a needle catheter which includes a needle.

The catheter assembly 200 may be used for local delivery of bioagents, such as cells used for cell therapy, one or more growth factors used for angiogenesis, or vectors containing genes for gene therapy, to the left ventricle. In one embodiment, the catheter assembly 200 described may be used in delivering cell therapy for heart failure or to treat one or more portions of the heart which are ischemic or infarcted. The catheter assembly 200 uses coaxially telescoping catheters 210, 240, and 280, at least one or more being deflectable, to position a medical instrument at different target locations within a body organ such as the left ventricle. The catheter assembly 200 is flexible enough to bend according to the contours of the body organ. The catheter assembly 200 is flexible in that the catheter assembly 200 may achieve a set angle according to what the medical procedure requires. The catheter assembly 200 will not only allow some flexibility in angle changes, the catheter assembly 200 moves in three dimensional space allowing an operator greater control over the catheter assembly's movement.

In one embodiment, one catheter in the catheter assembly 200 includes a deflectable portion. The deflectable portion allows the catheter assembly 200 the flexibility to bend according to the contours in a particular body organ. In one embodiment, the deflectable portion is a part of the first catheter 210. In an alternative embodiment, the deflectable portion is a part of the second catheter 240. In other alternative embodiments, both the first catheter 210 and the second catheter 240 may include deflectable portions.

Also, in certain embodiments, one of the first and second catheters includes a shaped portion which is a portion having a fixed, predetermined initial shape from which deflections may occur. For example, the second catheter 240 shown in one embodiment of the example of FIG. 6 includes, at its distal portion, a fixed, predetermined initial shape in which a first and second distal portion of the second catheter 240 form an initial angle which determines this initial shape. This initial angle may be between about 75 degrees to about 150 degrees. In the example shown in FIG. 6, the distal portion of the second catheter 240 has two portions which form a preshaped angle of about 90 degrees. The deflectable portion of the first catheter 210, in combination with the preshaped portion of the second catheter 240, allows for the distal tip of the third catheter to be selectively and controllably placed at a multitude of positions. It will be appreciated that the deflectable portion may alternatively be on the second catheter and the preshaped portion may be on the first catheter.

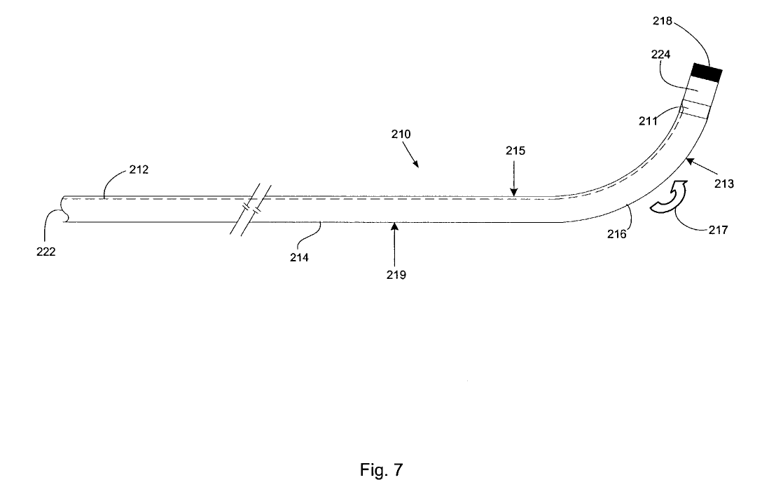

FIG. 7 illustrates a diagram of one embodiment of the first catheter 210 of FIG. 6. The first catheter 210 provides support and orientation direction to the other catheters 240 and 280. In one embodiment, the first catheter 210 provides support and orientation to the other catheters 240 and 280 across the aortic valve.

As shown in FIG. 7, the first catheter 210 includes a shaft with a proximal end 222 and a distal end 224. In one embodiment where the first catheter 210 includes a deflectable portion, the shaft is made up of a stiffer portion 214 and a deflectable portion 216 as shown in FIG. 7. The difference in stiffness may be achieved by having a wire braid reinforcement along the stiff portion and no wire braid reinforcement along the deflectable portion; other ways to achieve this difference include using different materials in the two portions. The location 215 shows, in one exemplary embodiment, the transition area between the stiffer portion 214 and the deflectable portion 216; as noted herein, this transition may be achieved by having a reinforcement layer or material in one portion and not having this layer or material in the other portion. It will be appreciated that both the stiffer portion 214 and the deflectable portion 216 are normally flexible enough to allow both portions to pass through a patient's vasculature (e.g. from an entry point into the femoral artery to a destination within the left ventricle or within a coronary artery). In an alternative embodiment where the first catheter 210 does not include a deflectable portion, the shaft may be made up entirely of a stiffer portion 214 which resists deflection.

In one embodiment, the first catheter 210 may also include a soft distal tip 218 at the distal end 224 of the shaft. The soft distal tip 218 can be a soft polymer ring that is mounted at the distal end 224 of the first catheter 210 to reduce trauma incurred as the catheter assembly 200 moves through the body.

In one alternative embodiment, the first catheter 210 may be made to have different preshapes. The preshapes allow the first catheter 210 to enter into specific body cavities and rest in preset positions. For example, once it is delivered into the ventricle, the first catheter 210 with a certain preshape rests in the ventricle with preferential positioning. The preshape typically includes at least one preset angle between portions of the first catheter; in the example of FIG. 6, the two portions define an obtuse angle.

In one embodiment, the outer diameter of the first catheter 210 is approximately 8 french or less. This is the case if the second catheter 240, not the first catheter 210, includes the deflectable portion. If the deflectable portion is on the first catheter 210, then the outer diameter of the second catheter 240 is 6 french. In one embodiment, if the deflectable portion is on the second catheter 240, then the outer diameter of the second catheter 240 will be 7 french.

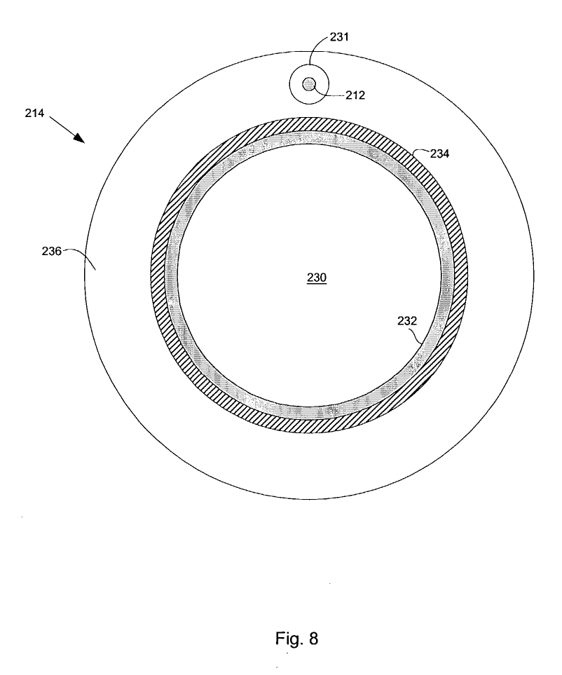

FIG. 7 also illustrates a pull wire 212. Pull wire 212 may be located inside a lumen (e.g. lumen 231 shown in FIG. 8) that runs along the first catheter 210. The pull wire 212 is attached to an anchor band 211 near the soft distal tip 218. When the pull wire 212 is pulled, the deflectable portion 216 bends as shown by arrow 217. In one embodiment, the tubing that houses the pull wire 212 may be made out of PTFE (PolyTetraFluoroEthylene or teflon). In an alternative embodiment the tubing that houses the pull wire 212 may be made out of any other flexible polymer.

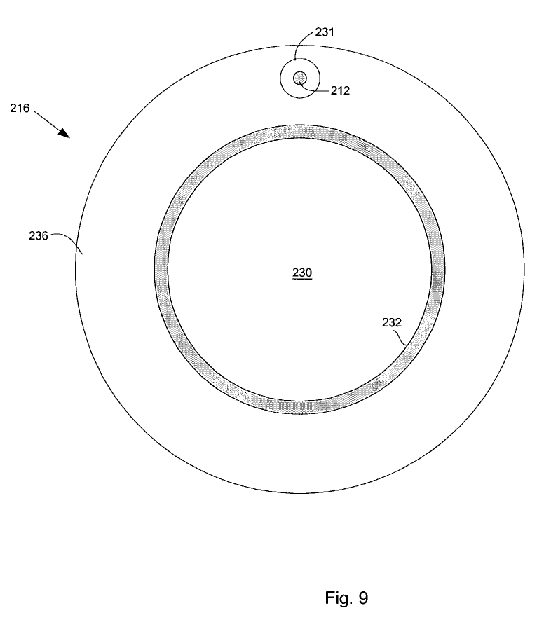

FIG. 8 illustrates a cross-section of the stiff portion 214 (taken at location 219) of the first catheter 210 shown in FIG. 7. As shown in FIG. 8, the stiff portion 214 of the first catheter 210 includes a liner 232, a braided reinforcement 234, and a jacket 236. The jacket 236 includes a lumen 231, formed in the jacket 236, and the pull wire 212 passes through lumen 231 as shown in FIGS. 8 and 9. In one embodiment, to build the stiff portion 214 of the shaft 220, a mandrel is inserted inside of the liner 232 for support. The liner 232 may be made of PTFE (PolyTetraFluoroEthylene or teflon) to produce a lubricious inner lumen surface. The interior lumen 230 of the liner 232 is designed to hold the second catheter which coaxially fits within this lumen of liner 232. The outer surface of the PTFE liner is chemically etched to promote adhesion with other materials. Next, a reinforcement material 234 is fabricated onto an outside layer of the liner 232. In one embodiment, the reinforcement material 234 may be braided. The reinforcement material 234 may be one layer or multiple layers. Next, a jacket 236 is attached to the outside of the reinforcement material 234. Shrink tubing (not shown) is wrapped around the outside of the jacket 236 and heated. The shrink tubing will shrink down and cause the other materials to be pushed inward in a fusing process. Accordingly, the jacket 236 will melt, penetrating the braid 234, if the reinforcement material 234 is a braided structure, and attach to the reinforcement material 234.

FIG. 9 illustrates a cross-section of the flexible portion 216 (taken at location 213) of the first catheter 210 shown in FIG. 7. The flexible portion 216 is similar to the stiff portion 214 but does not include the reinforcement material 234 of FIG. 8. Instead the flexible portion 216 includes the liner 232, lumen 231, pull wire 212 in the lumen 231, and the jacket 236 wrapped around the liner 232. The outer diameter of the cross-section of the portion 216 may be less than the outer diameter of the cross-section shown in FIG. 8. The absence of the reinforcement material at the distal portion of the first catheter allows this distal portion to be more flexible than a proximal portion of the first catheter. When the pull wire 212 is pulled, the distal portion deflects while the stiffer proximal portion deflects very little.

In one embodiment, the flexible portion 216 may include a second type of reinforcement material layer (not shown) between the liner 232 and the jacket 236. The second type of reinforcement material would be far less stiff than the reinforcement material 234 of the stiff portion 214. This second type of reinforcement material may be a metallic multi-ring structure to help maintain the lumen's opening (e.g. the lumen 230) when this portion of the catheter is deflected. It is noted that FIGS. 8 and 9 do not show the second and third catheters within the lumen 230.

In the process of making first catheter 210, the mandrel which is inserted into lumen 230 may be made of wire. In an alternative embodiment, the mandrel may be a glass filled polymer. In another alternative embodiment, the mandrel may be made of other materials, such as polymeric materials that can withstand heat (e.g. such that the material does not melt) when heat is applied to the shaft during the fusing process.

In one embodiment, the reinforcement material 234 may be made with stainless steel. In an alternative embodiment, the reinforcement material 234 may be made with nickel titanium wires. In another alternative embodiment, the reinforcement material 234 may be made with nylon wires. In other embodiments (not shown), the reinforcement material may not be braided. Instead of braiding, coils may be used.

In one embodiment, the tubing that houses the pull wire 212 may be placed between the liner 232 and the reinforcement material 234. In an alternative embodiment, the tubing may be placed between the reinforcement material 234 and the outer jacket 236. In that case, a first layer of reinforcement material 234 may be underneath the tubing with the pull wire 212, and a second layer of reinforcement material may be on top of the tubing with the pull wire 212. In another embodiment, multiple pull wires, in corresponding lumens in the jacket 236, may be used to control deflection of the first catheter.

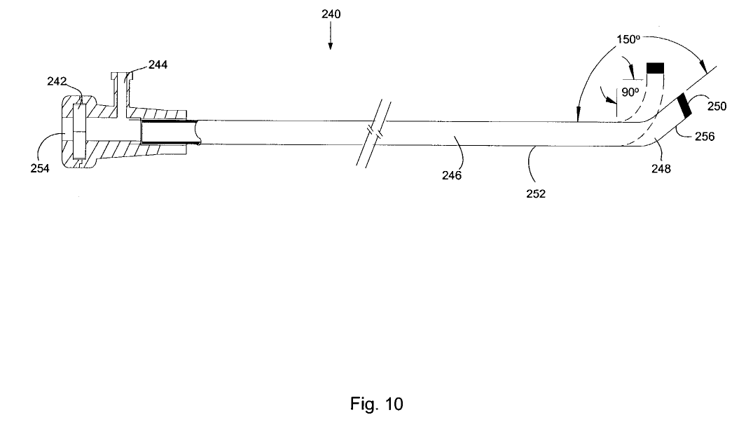

FIG. 10 illustrates a diagram of one embodiment of the second catheter 240 of FIG. 6. As discussed above, the second catheter 240 may include a flexible portion in one embodiment. In an alternative embodiment, the second catheter 240 may not include a flexible portion. In the embodiment shown in FIG. 10, the second catheter 240 includes a shaft 252 having a proximal end 254 and a distal end 256. The shaft 252 includes a stiffer portion 246 and a portion 248 which may be a flexible portion or it may have a predetermined initial shape. If the portion 248 has a predetermined initial shape, it may also be deflectable from this initial shape. The shaft construction of the second catheter 240 is similar to the first catheter 210 but may be made of material with relatively softer durometer. In one embodiment, the shaft 252 also includes a soft distal tip 250 (e.g., formed from a very low durometer material).

In one embodiment, the second catheter 240 may include a flush port 244 and a self-seal valve 242. The self-seal valve 242 ensures that fluid does not flow between the second catheter 240 and the third catheter 280. The flush port 244 allows flushing of fluid at any time. In an alternative embodiment, the first catheter 210 may also include a self-seal valve and a flush port. The flush port 244 may also be used to inject contrast media into the body organ to allow visualization of the body cavity.

In one embodiment, the distal end 256 of the second catheter 240 has a predetermined initial shape. This predetermined initial shape is typically an angle formed between two portions of this distal end. The distal end 256 of the second catheter 240 may be designed to provide support to the third catheter 280 through this predetermined shape. The shape will allow the second catheter 240 to direct the third catheter 280 to a target (e.g. see FIG. 6). In one embodiment, an angular range for the shaped distal end 256 of the second catheter 240 is approximately in the range of between 0° to 150°. In the case of FIG. 10, two exemplary angles of 90° and 150° are shown.

In one embodiment, where the portion 248 is deflectable, second catheter 240 is approximately a maximum of 10 centimeters in length longer than the first catheter 210. On the second catheter 240, the deflectable portion is no more than approximately 8 centimeters. The third catheter 280 extends less than 8 centimeters from the end of the distal end of the second catheter 240. In one embodiment, the third catheter extends 1 or 2 centimeters. The length of the third catheter 280 is dependent on the width and length of the heart. It will be appreciated that different sizes may be used, and these different sizes would normally be determined by the size of the organ which is intended to receive the catheter.

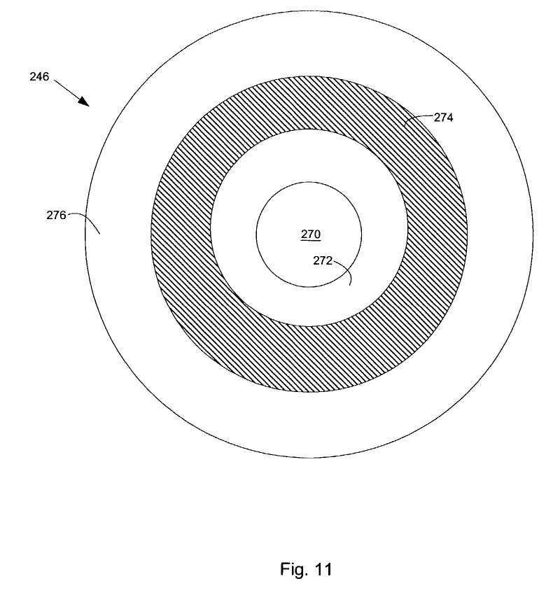

FIG. 11 illustrates a cross-section of the stiff portion 246 of the second catheter 240 of FIG. 10. Similar to FIG. 8, the stiff portion 246 includes a liner 272. The liner 272 has a hollow core which is the lumen 270 which is designed to coaxially receive the third catheter which is rotatably and slidably movable within the lumen 270. A reinforcement material 274 is fabricated onto the liner 272. A jacket 276 circumferentially surrounds the reinforcement material 274. Shrink tubing (not shown) is placed around the jacket 276. Heat is applied, and the shrink tubing shrinks to cause the reinforcement material 274 (e.g. wire braid) to become attached to the liner 272. The jacket 276 also then becomes attached to the reinforcement material 274. If the reinforcement material is a braided structure, the jacket material 276 may penetrate through the reinforcement material 274 and become attached to the liner 272.

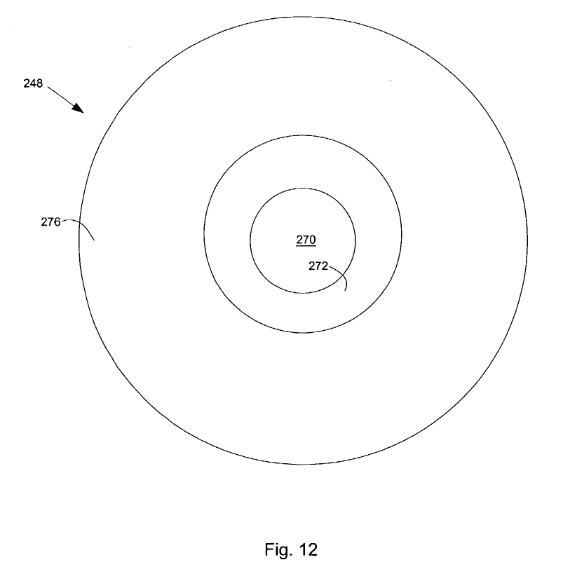

FIG. 12 illustrates a cross-section of the portion 248 of the second catheter 240 of FIG. 10. The cross-section is similar to the cross-section of FIG. 11 except that the portion 248 does not include a reinforcement material 274. Instead the portion 248 includes a liner 272 and a jacket 276 circumferentially surrounding the liner 272. In alternative embodiments, a second type of reinforcement material (not shown) may be etched or placed between the liner 272 and the jacket 276 for the portion 248. This second type of material may be a metallic multi-ring structure to help maintain the lumen dimension (e.g. the opening of the lumen) when this portion 248 of the catheter 240 is deflected (if it is deflectable).

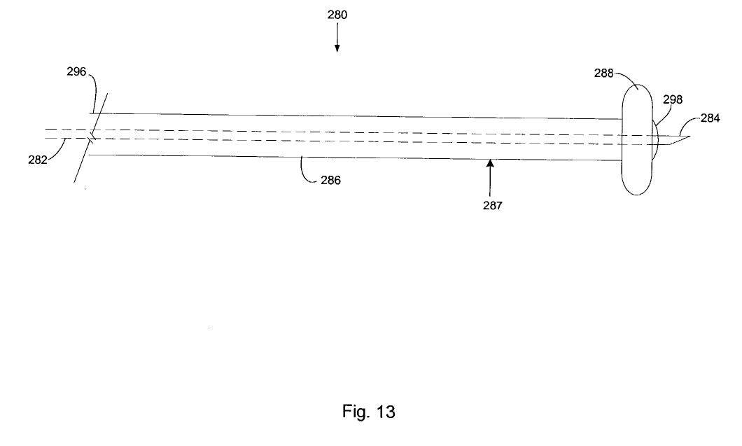

FIG. 13 illustrates a diagram of the third catheter 280 in FIG. 6. The third catheter 280 guides a medical instrument, such as a needle, to a target area. In one embodiment, the third catheter 280 may be a needle catheter as seen in FIG. 13. The third catheter 280 includes a needle sheath 286 housing a needle 282. The needle is movable longitudinally through the sheath 286, and the lumen of the needle extends from a proximal end of the needle to the needle tip 284. The needle sheath 286 has a proximal end 296 and a distal end 298. A needle tip 284 of the needle 282 is extendable from the distal end 298 of the needle sheath 286 (as shown in FIG. 13). While the needle 282 is shown as a straight needle with a sharp tip, other types of needles, such as helical (e.g. corkscrew-like) needles may also be used in certain embodiments.

In one embodiment, the outer diameter of the needle sheath 286 is between 40 to 60 thousandths of an inch. In one embodiment the needle 282 is a 25 to 27-gauge needle. This may be the case if the outer diameter of the first catheter 210 is approximately 8 french. The outer diameter may change if the diameter of the first catheter 210 increases.

In one embodiment, the third catheter 280 may include one or more stabilizers. As seen in FIG. 13, the stabilizer in one embodiment is a balloon 288. The balloon 288 is located near the distal end 298 of the needle sheath 286. The balloon 288, in this case a tire tube shaped balloon, allows the third catheter 280 to approach the target with the needle 282 perpendicular to the target. In other words, the tire tube shaped balloon will tend to prevent a non-perpendicular needle injection into the target tissue. In addition, the balloon 288 allows for a large surface area of control so the needle tip 284 or needle 282 does not wobble. For example, as the third catheter 280 approaches a wall of the left ventricle, the balloon 288 is positioned against the wall of the left ventricle. The needle 282 then extends from the sheath 286 and penetrates the left ventricle wall. The balloon 288 thereby allows for a larger surface area of control against the left ventricle wall to stabilize the needle 282 and hold the needle 282 perpendicular to the left ventricle wall as it penetrates through the surface of the wall.

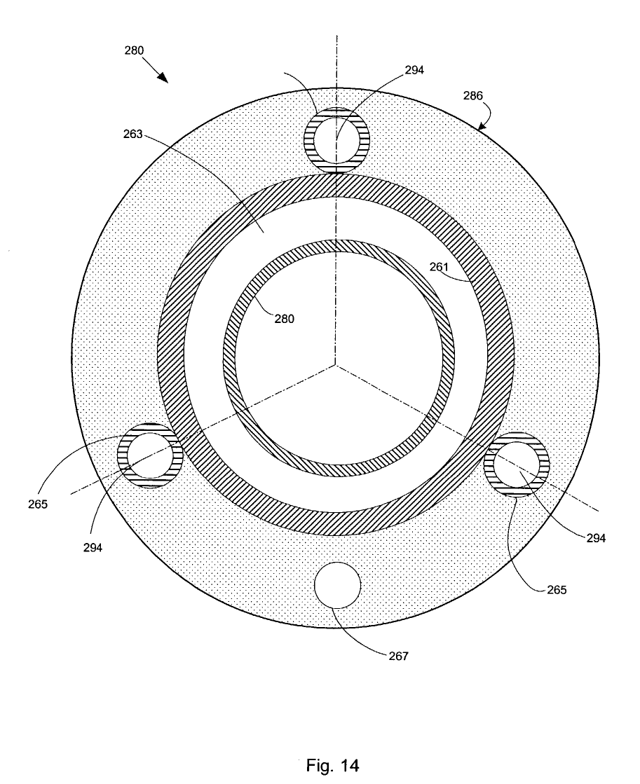

FIG. 14 illustrates a cross-section (taken at point 287) of the third catheter 280 of FIG. 13. In one embodiment, and as shown in FIG. 14, three balloon lumens 294 are placed between the needle 282 and the outer layer of sheath 286. Each balloon, such as balloon 288, may use a separate balloon lumen 294. In one embodiment, one balloon lumen 294 may be used with one balloon stabilizer. In alternative embodiments, additional balloon lumens 294 may be used for only one balloon stabilizer or for more than one balloon stabilizer. In FIG. 14, the three balloon lumens 294 are positioned relative to the sheath 286 at various points to provide additional strength to the structure of the third catheter 280. This additional strength allows for additional stabilization and nonbuckling of the third catheter 280. In one particular embodiment, shown in FIG. 14, the three balloon lumens 294 are coupled to a single tire tube shaped balloon 288 which is attached to the distal end of the third catheter 280 as shown in FIG. 13. These three balloon lumens 294, when inflated, tend to give additional strength to the third catheter. These three balloon lumens 294 are arranged substantially equidistant in an angular manner relative to the outer circumference of sheath 286 in order to provide a substantially equal distribution of support to the third catheter; in particular, they are separated by about 120 degrees. These lumens 294 are created by tubular liners 265 which are embedded, in one embodiment, into the sheath 286. Another tubular liner 261 forms the lumen 263 which slidably receives the needle 282. Lumen 261 extends from the distal end of the third catheter 280 to the proximal end of the third catheter 280. Lumens 294 extend from a point at which they are coupled to balloon 288 (near the distal end of the third catheter 280) to a proximal end of the third catheter whereat these lumens 294 are coupled to a source for an inflation fluid which is used to inflate balloon 288. Lumen 267 is an optional lumen for use with a pull wire (not shown) which may be used to deflect the third catheter 280 in certain embodiments.

To precisely show the position of the catheter tip relative to the heart, a plurality of cardiac images are recorded and gated according to one or more cardiac parameters, such as electrocardiogram (ECG), heart sounds, pressure, ventricular volume, and others. When the recorded images are played back according to the real time measurement of these cardiac parameters, the real time position of the catheter tip relative to the heart can be precisely displayed.

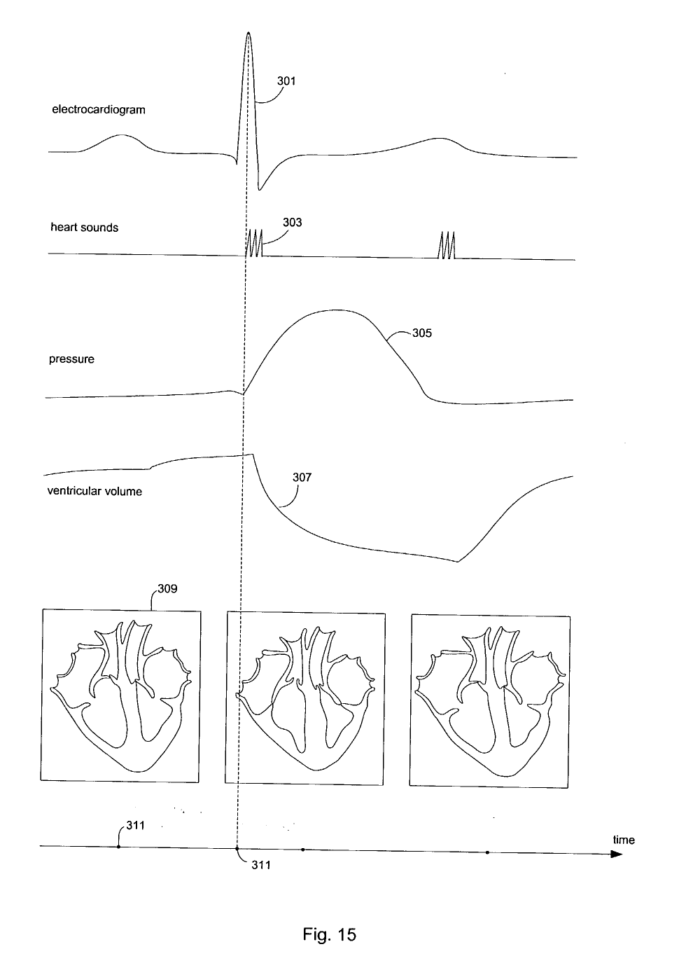

FIG. 15 illustrates various methods to prepare images for guiding real time position tracking according to embodiments of the present invention. According to one embodiment of the present invention, the images obtained at various instances in the cycle of a heartbeat are associated with the time after a specific feature of the cycle, indicated by a parameter. For example, electrocardiogram 301 can be taken concurrently with the process of scanning the patient for the cardiac images (e.g., image 309). From comparing the timing of the occurrence of the specific feature (e.g., “R” wave at time 313) and the timing of the image generation (e.g., time 311 for image 309), the images of the heart can be correlated with the instances of time after the occurrence of the specific feature (e.g., “R” wave).

When the heart rate is not arrhythmic and doesn't vary greatly during the scanning process, images obtained from multiple cycles can be mapped into various instances in a single cycle, relative to the specific feature. The heart is at its most repeatable positions based on the time of ventricular contraction (time after ECG “R” wave for ventricular imaging or time before “R” wave for atrial imaging). When the heart rate is not arrhythmic, but varies greatly during the scanning process, different single cycles may be created for individual heart rate ranges. This may require several scanning processes to fully collect the desired imaging data, but may be necessary for patients with unstable heart rates. However, in the case of arrhythmia (e.g. a PVC, Premature Ventricular Contraction), the images collected in this period can be discarded, as well as the images from the next cardiac cycle. After the heart recovers and returns to a more normal contraction/motion, the positions of the heart will be more repeatable.

Other cardiac parameters (e.g., heart sounds 303, pressure 305, ventricular volume 307, and others) can also be used to gate the cardiac images. For example, pulmonary artery pressure can be used at least as one of the parameters to correlate with the recorded images. The flow-directed balloon-tipped pulmonary artery (PA) catheter, also known as the Swan-Ganz catheter (SGC), has been in clinical use for almost 30 years. Initially developed for the management of acute myocardial infarction (AMI), it now has widespread use in the management of a variety of critical illnesses and surgical procedures. Anesthesiologists typically use it to monitor the condition of their patients during surgery. It is usually used to measure: cardiac output, pulmonary artery pressures and pulmonary wedge pressure (about the same pressure that would be measured in the left atrium). Examples of discussions related to Swan-Ganz catheters can be found in: J. Thorac Cardiovase Surg, vol. 71, no. 2, 250-252, 1976; Cardiovasc Clin, vol. 8, no. 1, 103-111, 1977; and, Clin Orthop, no. 396, 142-151, 2002.

Further, other parameters that characterizing the state of the heart can also be used for gating the playback of the pre-recorded images. For example, relative wall motions of a heart can be measured in a CT or MR imaging system to correlate with the state of the heart. Real time relative wall motion can be determined using a 3D position determination system (e.g., by keeping the mapping catheter tip in contact with the wall of the heart). Thus, the pre-recorded images can be played back according to the wall movement of the heart.

In one embodiment of the present invention, images obtained from one or more cycles with the concurrently measured cardiac parameters are used to construct a mapping between measured cardiac parameter and the cardiac images. For example, the images can be correlated to the ECG level (e.g., for a specific portion of the heartbeat cycle); thus, a measured ECG level can be used to determine the corresponding cardiac image. In one embodiment of the present invention, a heartbeat cycle is divided into a number of segments, according the features (e.g., the occurrence of maximum and/or minimum points, etc.) so that the time can be normalized for each segments individually; and, within each segment, different cardiac images can be constructed as functions of one or more cardiac parameters.

In one embodiment of the present invention, the hemodynamic state of the patient is stable and similar during the imaging operation and during the therapy process so that the image selected or generated from the correlation between the measured cardiac parameters and the pre-recorded images accurately represents the real time state of the heart. In such an embodiment, care is taken to ensure that the patient's hemodynamic state (e.g., blood pressure, heart rate, hydration state, blood volume, cardiac output, sedation state, ventilation state, respiration state, or others) during the 3-D imaging and during the therapy guidance is similar. For example, in both operations, the patient will be supine. Also, the patient is in similar sedation states; and, the time interval between imaging and therapy is minimized such that the disease state does not progress significantly (e.g., causing significant cardiac dimensional changes).

In another embodiment of the present invention, the imaging operation is performed for a number of different hemodynamic states (e.g., blood pressure, heart rate, hydration state, blood volume, sedation state, ventilation state, respiration state, or others) so that the pre-recorded images can be selected or corrected (e.g., using an interpolation scheme) according to the real time hemodynamic state.

In a typical process to obtain diagnostic images, a patient is instructed to breathe shallowly or to hold the breath during an imaging run, since the chest movement can induce changes in the position and shape of the heart. According to one embodiment of the present invention, the patient's ventilation parameters and/or chest position/movement is also simultaneously monitored and recorded during the imaging run so that the cardiac images can be corrected or correlated with the breathing of the patient.

A calibration method is used to ensure that the coordinate system of the location system and the recorded images are properly overlaid. Some examples are described below. However, it is understood that the details of the calibration are open to many permutations and are dependent upon the modalities used.

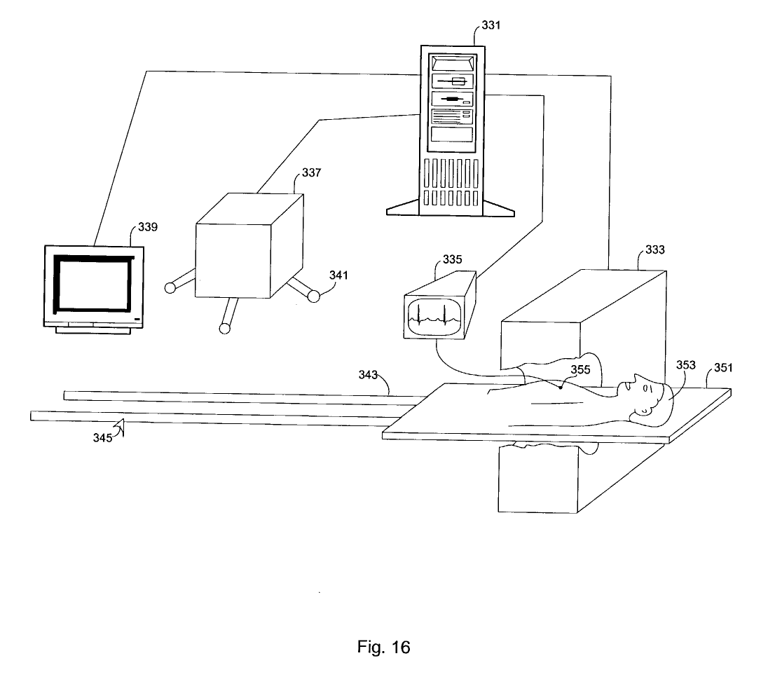

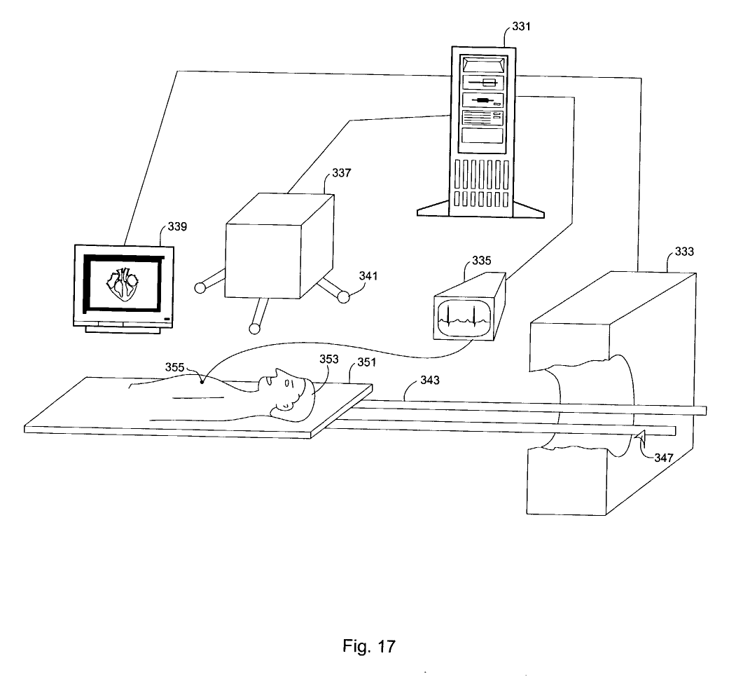

FIGS. 16-17 illustrate a method to align coordinates of a position tracking system with coordinates of an imaging system according to one embodiment of the present invention. In FIG. 16, patient 353 is in an imaging system (e.g., a CT or MRI system) for the generation of images. Patient 353 is secured on operation platform 351, which has a known position relative to the imaging system. Device 335 collects ECG (or other parameters, such as cardiac parameters, hemodynamic parameters, ventilation parameters and/or chest position/movement) through sensor(s) 355, while imaging system 333 obtains cardiac images of patient 353. Both measured parameters and obtained images are stored on data processing system 331, which correlates the measured parameters with the images while images are being obtained or after the imaging operation is finished. The images can be enhanced to show the areas of interest (e.g., the ischemic regions). Such enhancement can be performed using data processing system 331 or other data processing system (e.g., connected through a communication link or a computer network).

In one embodiment of the present invention, rail 343 is used to transport the patient from imaging system 333 to position tracking system 337 (e.g., catheter laboratory) without moving the patient relative to operation platform 351. Positioning device 345 is used to align platform 351 with respect to the position tracking system (e.g., when device 345 locks onto a specific portion of the operation platform 351). For example, when platform 351 is moved toward device 345 from imaging system 333 along rail 343, device 345 stops and looks platform 351 at a predetermined position. It is understood that various devices known to the person in the art can be used to physically align (or lock) the operation platform with respect to imaging system 333 (e.g., using device 347 in FIG. 17) at one position and with respect to position tracking system 337 in another position. Detailed implementation of devices 345 and 347 is not germane to the present invention.

In FIG. 17, operation platform is aligned respect to position tracking system system 337 (e.g., using device 345 as illustrated in FIG. 16). Patient 353 remains to be secured to the operation platform. Sensors 355 collect data for generating the same type of parameters (e.g., ECG) in device 335, which is used by the data processing system to generate (e.g., selecting from the recorded images or creating from the recorded images through interpolation, or others) to display cardiac images real time according to the real time measured parameters. The images are displayed on display 339 at real time according to the signals for sensor 355 to provide an illusion of displaying real time cardiac images.

In one embodiment of the present invention, position system 337 has a number of signal generators (or sensors) installed at a number of locations (e.g., 341). When a sensor (or generator) is attached to an instrument (e.g., a catheter tip), the position (and the orientation) of the instrument can be determined by position tracking system 337. For example, acoustic, magnetic or radio frequency based position tracking systems can be used to determine the position of the instrument. A radio frequency based position determination system (e.g., Global Positioning System, a local positioning system using the same clock in both the transmitter and the receiver) using the signal delay detected in transmitting along different paths between the tracked object and each of a number of reference points. Further, optical systems (e.g., using low frequency, Infrared (IR), or high-strength light with sensors to detect 3 or more light sources) may also be used for determining the position of the instrument.

The position system determines the position of the instrument relative to the generators (or sensors) (e.g., 341); and, the images are generated relative to imaging system 333. When the positions of operation platform 351 relative to imaging system 333 in imaging and relative to position system 337 in position tracking are determined, transformations for representing the data spatially relative to operation platform 351 can be determined mathematically, using methods known in the art. Since the patient is fixed relative to the platform, the transformations can be used to determine the tracked position relative to the heart depicted in the pixel images from the imaging system. After determining the position of the tracked device relative to the heart, a representation of the device (e.g., a catheter tip) can be superposed on the image on display 339 to show the device relative to the heart depicted in the image.

In one embodiment of the present invention, the position of operation platform 351 relative to position tracking system 337 at one reference location is known to the system (e.g., through an installation procedure). In another embodiment, operation platform 351 is not locked at the reference location during a cardiac therapy. One or more sensors can be used to measure, sense, or determine the current position of the operation platform relative to the reference location so that the system can effectively determine the tracked position relative to the heart represented in scanned images (e.g., adjust the tracked position to obtain the coordinates that corresponding to those when the platform is at the locked at the reference location). For example, in the position determination system, the platform's current position information can be used to adjust the coordinate values of the tracked position. For instance, if the platform in the position determination system is at the positive X axis direction 127 mm from the reference location, the X axis position of the tracked position can be subtracted by 127 mm in the X axis. Thus, the physician can position the operation platform at a convenient location for the therapy operation. Similar, sensors can be used to determine the position of the platform relative to a reference position, when the platform is attached to the imaging system for imaging. Various instruments for sensing or measuring the position of the platform relative to reference positions can be used. In one embodiment of the invention, the measurement of the position of the platform relative to the reference position is automatically performed, so that the data processing system 331 can automatically adjust the transformation to superpose the tracked position on the images with respect to the heart.

Although the above description illustrates an embodiment where the platform is transported with the patient, the platform can be transported separately from the patient in another embodiment. Provisions (e.g. adjustable pegs under the armpits on the platform, adjustable foot position holders on the platform, adjustable head position holders on the platform, adjustable hip position holders on the platform etc.) can be made such that the patient is placed on the platform in the position determination system in virtually the identical position in which the patient was during the collection of the cardiac images in the imaging system. In a further embodiment, distinct platforms are used in the position determination system and in the imaging system. Similar provisions are made such that the patient's positions (and/or orientations) on the platforms (in the position determination system and in the imaging system) are identical or known (e.g., through sensors attached to such provisions) so that the positioning of the patient on the platform (platforms) is controlled in essentially the same way as using a single platform.

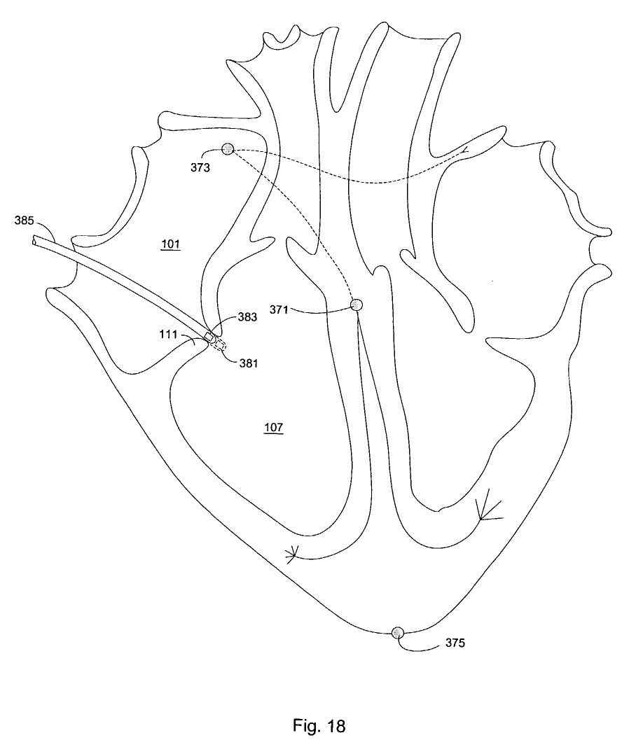

FIG. 18 illustrates alternative methods to register coordinates of a position tracking system with coordinates of an imaging system according to embodiments of the present invention. In one embodiment of the present invention, at least four reference points in one of the images at known anatomical and/or spatial positions relative to the patient or a known reference frame are used to align the coordinate systems of the imaging system and the position tracking system. When more than four reference points are used, a least square procedure (or other mathematical matching algorithms) can be used to determine a best alignment. By identifying these reference points in the coordinate systems of both the imaging system and the position tracking system, a mathematical transformation can be determined to map the tracked position relative to the reference points to the corresponding locations in the images relative to the corresponding reference points. For example, sinoatrial (SA) node 373 in the right atrium, as shown in FIG. 18, generates activation signal for initiating contraction of muscle fibers. Atrioventricular (AV) node 371 delays the activation signals from the SA node to activate the contraction of ventricles. SA node and AV node can be identified by using a catheter that measures the electrical physiological values at the tip of the catheter. When the catheter tip reaches the SA node or the AV node, the position of the catheter tip in the images from the imaging system can be identified on the images. When the patient is in the position tracking system (e.g., in Cath Lab), the position of the catheter tip, which is in contact with the SA node or the AV node, can be determined in the position tracking coordinate space. After three or more fiducial points are identified in both the imaging coordinate space and the position tracking coordinate space, a transformation can be derived mathematically to overlay the position tracked on the images from the imaging system to show the tracked position relative to the heart using various mathematical formulations known in the art. In additional to the AV and SA nodes, other anatomical or spatial reference points (e.g., apex 375, tricuspid valve 111, entrances to coronary arteries, entrances to coronary sinus, aortic valve, pulmonary valve, and others) can be used. For example, the position of the tricuspid valve can be identified using a pressure sensor at catheter tip 383. When the catheter tip is slowly moved from right ventricle 107 toward right atrium 101 (e.g., from position 381 toward position 383), the pressure detected by the sensor changes. Since there is a change in pressure across the tricuspid valve, the catheter tip can be placed at (or near) the tricuspid valve by monitoring the measured pressured.

Different means can be used to determine the position of the fiducial points in the imaging system and the position determination system. For example, fiducial points can be marked (e.g., with ink). Radiopaque markers can be used at the marked fiducial points to mark the positions of the fiducial points in the imaging system. After the patient is moved to the catheter laboratory, magnetic coils (sensors or signal generators) can be placed on the marked fiducial points (instead of the radiopaque markers) to identify the fiducial points in the position determination system.

In one embodiment of the present invention, the fiducial points are located outside the heart or organ of interest. For example, fiducial points can be on the chest of the patient. Further, the fiducial points can be on the operation platform so that the imaging coordinate space and the position tracking coordinate space are aligned with respect to the operation platform at reference positions (e.g., before the patient is placed on the operation platform). Once the patient is secured relative to the operation platform, the transformation for align the imaging coordinate space with the position tracking coordinate space with respect to the operation platform can be used to superpose the tracked position on the imaging from the imaging system with respect to the heart of the patient.

Thus, reference points and/or orientations of an organ/body that are identifiable both on the images recorded in the imaging system and in the position determination systems can be used in aligning the coordinate systems. The reference points may be anatomical locations (e.g., landmarks, such as the ventricular apex, a coronary ostium, vessel branch points, etc.) and the orientations may be indicated by anatomical features (e.g. the spine, a blood vessel, a line connecting two anatomical locations). An object or a number of objects can be attached to the organ/body to identify the reference points and/or orientations of the anatomy in the images and in the position determination systems. If the reference points and/or orientations appear in a recorded image (e.g., when the objects are opaque to X-ray), these reference points in the imaging system are known. Alternatively, the reference points and/or orientations of the anatomy in the imaging system are determined by means of another measurement system linked to the imaging system. Prior to overlaying the position of the medical device on the image(s) of the organ/body, the reference points and/or orientations of the anatomy relative to the coordinate system of the positioning system are recorded. These reference points and/or orientations can be recorded by positioning the medical device and/or some other portion of the position determination system at the reference points. The object (or objects) used to identify the reference points and/or orientations of the anatomy in the imaging systems can be different from the one used to identify the reference points and/or orientations of the anatomy in the position determination systems.

The quality of alignment in cardiac applications can be greatly improved by gating the reference point and/or orientation data relative to a time related cardiac parameter (such as the ECG or a blood pressure waveform) such that the reference points and/or orientations used are at the same or nearly at the same point in the cardiac cycle. Similarly, the quality of the alignment (as well as the location accuracy of the overlay) may be improved by gating the image data collections and the position/orientation data collections in a similar manner and to the same time related cardiac parameter. The quality of the alignment may also be improved by assuring that the hemodynamic state of the patient is relatively unchanged during the recording of the reference points and/or orientations by the imaging system and by the position determination system. Monitoring and controlling such parameters as the patient's blood pressure, heart rate, respiration, hydration state and sedation state can be used to improve the quality of the alignment. Simultaneously gating to a respiratory parameter, such as chest motion or to the cycle of a respirator (if used), and a cardiac parameter can further improve the quality of the alignment. Additionally, ensuring that the patient's hemodynamic and respiratory parameters are relatively the same during the imaging recording and during the use of the position determination system to overlay the device's real-time location onto the recorded images improves the location accuracy of the overlay.

In general, at least four non-coplanar reference points, which are not in a same plane, are required to generate a transform to align two 3-D coordinate systems; and, at least three non-collinear references points, which are not in a straight line, are required to generate a transform to align two 2-D coordinate systems in a plane. When certain relations (e.g., orientation and/or scale) between the coordinate systems are known, fewer reference points can be used to align the coordinate systems. For example, when both the coordinate systems are aligned with the horizontal plane and aligned with one axis, a single out of plane reference point can be used to align the coordinate systems, if the same scale (unit of measurement) is used for the two coordinate systems. The quality of alignment can also be improved when more than the required points are used to determine a best-fit alignment transform (e.g., using mathematical algorithms for optimization known to the person skilled in the art). The collections of reference points represent geometric features, such as lines, curves, planes, or other higher dimensional objects and angles. For example, during an imaging sequence, a suitable dye may be injected into a coronary artery, allowing a good image of the coronary artery to be recorded. Between two anatomical landmarks, such as vessel branches, a set of points forming a curved line of the coronary artery through the middle of the lumen can be collected in the imaging coordinate system. The medical device (e.g., a catheter) is inserted (e.g., under fluoroscopic guidance) in the same artery; and, the locations of the device in the position determination system can be recorded along the same segment of the coronary artery. A transform is then generated from matching the two curves that are represented by the sets of points determined in the coordinate systems of the imaging system and the position determination system. Further, the positions of the collections of reference points can be gated according to the cardiac cycle in a cardiac application. For example, the recorded coronary artery images are resolved into sets of points describing curved lines of the vessel branch in the imaging system's coordinate system at a number of points in the cardiac cycle. Similarly, the positions of the points along same segment of the coronary artery in the position determination system are determined (e.g., from the tip position of the inserted catheter) at the corresponding points (or different points) in the cardiac cycle. The location data points from the curves corresponding to the same or nearly the same point in the cardiac cycle provide ample data to create an alignment transform. From this description, a person skilled in the art can envision the wide variety of alternatives and combinations of alternatives in the collection, interpolation and pairing of the reference location data needed to create the alignment. The best alternative will be in general governed by such factors as the imaging recording modality, the position determination system modality, medical device design, the medical procedure's positioning accuracy and repeatability requirements, the physician's device positioning experience and the physical state of the patient.

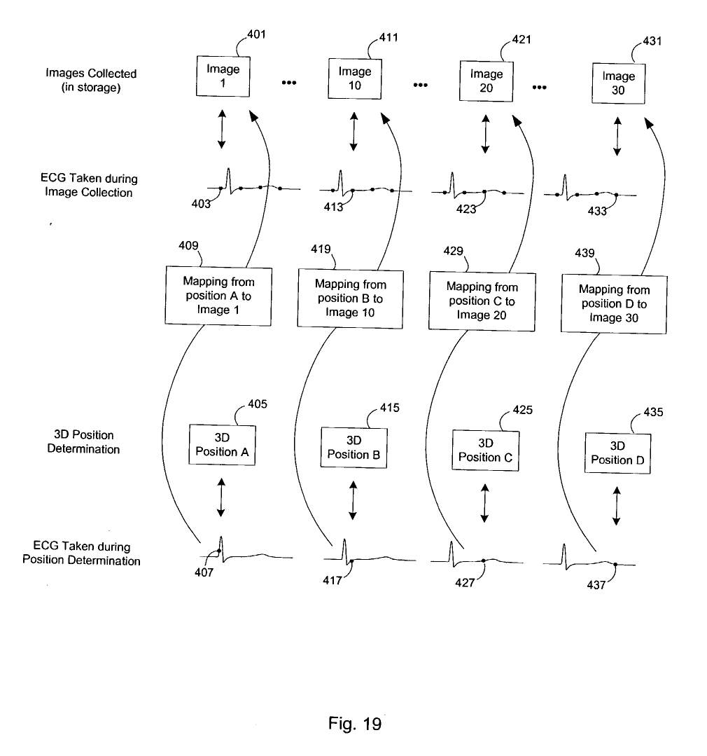

FIG. 19 illustrates a method to map real time tracked positions to corresponding pre-recorded images according to one embodiment of the present invention. In FIG. 19, images 401, 411, 421 and 431 represent images collected from an imaging system (e.g., a CT or MRI system). Data 403, 413, 423 and 433 represent the ECG taken during the collection of images 401, 411, 421 and 431 respectively. Data 405, 415, 425, 435 represent the 3D position determined from a position tracking system; and, data 407, 417, 427 and 437 represent the ECG taken when the position data 405, 415, 425 and 435 are obtained. The collected images (e.g., 401) are correlated to the ECG taken during image collection (e.g., 403). When a position (e.g., 405) is determined and ECG (e.g., 407) is taken substantially contemporaneously, the ECG taken during the position determination is matched with the ECG taken during the collection of images. In one embodiment of the present invention, the image with the closest matched ECG is selected; and, an operation (e.g., 409) is performed to map the 3D position (e.g., 405) to the corresponding location in the recorded image (e.g., 401).

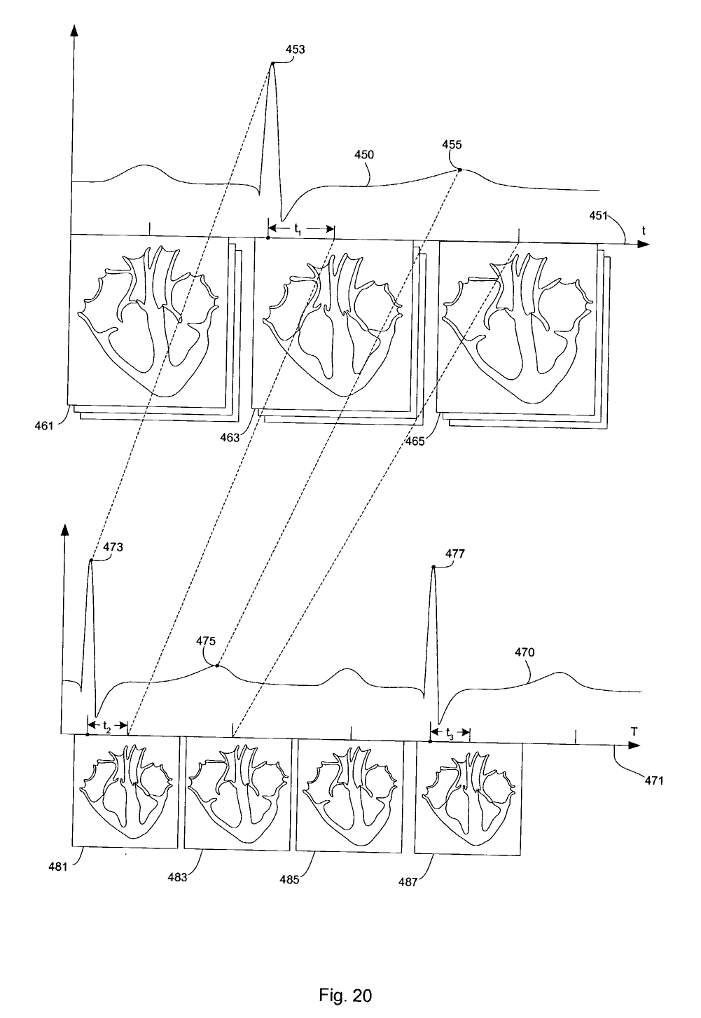

FIG. 20 illustrates another method to generate simulated real time cardiac images from pre-recorded images and real time measurements of cardiac parameters according to one embodiment of the present invention. In FIG. 20, timeline 451 represents the time relative to a specific feature (e.g., “R” wave 453). ECG 450 represents ECG collected when the images 461-465 are generated from the imaging system. Timeline 471 represents the time when ECG signal 470 is measured. Since feature 473 corresponds to feature 453, image 463 that is period t₁ after the occurrence of feature 453 is selected for display at a same period after the occurrence of feature 473. If the heart rate and other hemodynamic parameters did not change substantially since the image was obtained from the imaging system, then this image will be an accurate simulation of the actual real time cardiac anatomy. Similarly, other images are selected for display according to matching the timing of the features in ECG 470 and ECG 450. Since the heart rate may be changed after the images are obtained from the imaging system, appropriate scaling can be used to correlate the timing. For example, the timeline can be normalized with respect to the period of the heartbeat (e.g., t₂ is normalized with respect to the time period between the period between “R” waves 473 and 477 so that the normalized time is equal to t₂ normalized with respect to the heart beat cycle for timeline 451). In one embodiment of the present invention, ECG 470 is measured at real time. To display the image sequence in real time, the period of one or more previous cycles are used to predict the period of the current cycle, which is used to normalize timeline 471 for the current cycle. For example, the time period between “R” waves 473 and 477 can be used as the predicted heart beat cycle for determining time t₃ after “R” waves 477 to display image 487, which corresponds to image 463 after “R” waves 463. Further, additional features (e.g., maximum point 455 which corresponds to point 475) can be used to divide the cycle into multiple segments. Each of the segments can be scaled individually, according to the corresponding segments of the previous cycles. From this description, a person skilled in the art can envision various different methods for predicting the current heartbeat rate according to the activity in the previous cycles, using the time period of previous cycles and/or feature segments.

Since the heart is at its most repeatable positions based on the time of ventricular contraction (time after ECG “R” wave for ventricular imaging or time before “R” wave for atrial imaging), one embodiment of the present invention associates the time of ventricular contraction and the heart rate with the corresponding cardiac images so that the image that is corresponding to the real time measured heart rate can be selected for display at the corresponding time of ventricular contraction. For example, image 463 is associated with time t₁ after feature 453 as well as an indicator of the heart rate at the time the image is obtained (e.g., the time period between feature 453 and the corresponding one immediately before it). A set of images for the same time t₁ after feature 453 can be collected for different heart rates. The particular image (e.g., 487, in this case t₃=t₁) that is displayed at the real time interval is selected according to the time after the reference feature (e.g., 477) and the real time heart rate (e.g., as indicated by the time period between features 473 and 477). Images can be further selected for display in real time according to any relevant hemodynamic parameters, respiratory parameters or other parameters (or, alternatively, under the same or similar conditions to those parameters).

Cardiac images can also be collected according to a time after a feature (e.g., time t₁ after feature 473) for multiple planes through the heart. Thus, multiple slices of cardiac images at the given time after the specific feature represent a 3-D image matrix of the heart at the given time after the feature. The particular image slice (e.g., 481) at the corresponding time in the cardiac cycle after the corresponding feature (e.g., 473) is selected (or computed) according to the real time position information of a portion of the medical device (e.g., the slice closest to the position, or plane, of the portion of the medical device is selected). The particular image slice is then displayed with a representation of the portion of the medical device overlaid on it.

From this description, a person skilled in the art understands that some of the above-described methods can be combined in various ways. For example, the 3-D image matrix of heart can be generated for a time after a given feature for a number of heart rates or ranges of heart rate. Thus, the image selected for display at the real time depends on the real time heart rate, as well as the position of the portion of the medical device. A multidimensional image matrix can be collected and associated with various physiologic parameters or ranges of parameters (image pixel coordinates and, pixel intensity and associated physiologic parameters may each be considered a dimension of the recorded image matrix); and, the real time physiologic parameters and the position of the portion of the medical instrument can be used to determine the image for display. Further variations may be initiated and/or controlled by the operator and/or provided by the equipment manufacturer. For instance, the orientation of the planes of the image slices may be selected by the operator and/or determined to match the orientation of the portion of the medical device. In another instance, the recorded image matrices may be processed prior to medical device use to create/store 3-D surface matrices of interest (from the multidimensional image matrix) for use in later overlaying their projections and a projection of the portion of the medical device. Such an image may then be rotated under operator control to provide a visual sense of the 3-D relationships on a 2-D monitor screen.

In one embodiment of the present invention, the tracked positions are recorded as a function of time such that the positions of the tracked objected can be determined for the instance when an image is to be displayed. A representation of the tracked object is overlaid on the image for display substantially real time.

In one embodiment of the present invention, a real-time position of the portion of the device relative the anatomy (e.g., the real-time position of the catheter tip relative to the heart, as determined from the position tracked by the position tracking system and from the selected cardiac images according to the real time cardiac parameters) is recorded and annotated during a therapeutic or diagnostic operation, in addition to displaying the real-time position of the portion of the device relative to the anatomy. For example, the pre-recorded image matrix (or image data selected or processed based on the real-time condition) can be modified to record such a position; or, a modified copy of prerecorded image data or part of the image data (like time after ECG “R” wave=0 image data) can be created; or, data related to the original pre-recorded image and/or other data derived from the pre-recorded images can be stored in machine readable media to indicate the real-time position of the portion of the device relative to the anatomy; or, the real-time position can be recorded so that it can be displayed in various manners without the pre-recorded image(s). The annotation can be in terms of selected icons/symbols, a color coding, entered writing, the time and/or sequence of the annotation or annotation type, data from a catheter mounted sensor, data from another sensor or other equipment or derived data that indicate diagnostic or therapeutic information about that position and/or information gathered at the time or near the time that the device portion was at or near that position, or other forms and combinations of forms. This type of recording allows a procedure to be well documented for future review and analysis. It also allows the physician to more effectively guide a therapy by allowing other collected diagnostic information to be represented/accessible on/from the image(s)/display and, thus, it is easier for the physician to relate the collected diagnostic information to anatomic and/or other represented diagnostic information. It also allows the physician to more effectively guide a therapy by representing on the image(s) the locations and types of therapy previously applied. It may also be configured to display derived data from the previously recorded positions, real-time position data and/or annotations/annotation data (i.e. display the distance of the current real-time position of the portion of the device from the nearest previously recorded position that had a certain annotation), which would be especially useful in therapies requiring an injection at intervals over a selected tissue surface (spatial dosing). In another example, it may also be configured to display and/or record the change in position, maximum velocity and/or maximum acceleration of a recorded position over an ECG R-R interval or several intervals, which is a good indication of the contractile health of cardiac tissue.

In one embodiment of the present invention, interpolations are performed to provide intermediate frames of images from the collected images so that a smooth video image of the beating heart can be displayed according to the real time measured cardiac parameters, with a representation of the tracked object displayed at a position relative to the heart, according to the real time position information determined by the position tracking system.

It is understood that parameters related to the shape and position of the heart, such as chest position (and/or movement), hemodynamic parameters, ventilation parameters, and other cardiac parameters (e.g., blood pressure, pulse wave, heart wall motion), can also be used to gate the playback of the pre-recorded images. Indicators based one or more of these parameters can also be generated to gate the playback of the images.

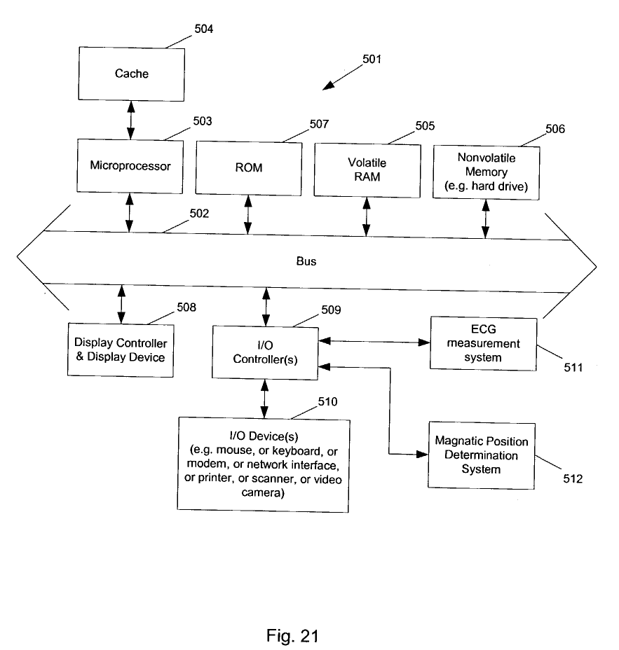

FIG. 21 shows one example of a typical computer system which may be used with the present invention. Note that while FIG. 21 illustrates various components of a computer system, it is not intended to represent any particular architecture or manner of interconnecting the components as such details are not germane to the present invention. It will also be appreciated that network computers and other data processing systems which have fewer components or perhaps more components may also be used with the present invention. The computer system of FIG. 21 may, for example, be an Apple Macintosh computer.

As shown in FIG. 21, the computer system 501, which is a form of a data processing system, includes a bus 502 which is coupled to a microprocessor 503 and a ROM 507 and volatile RAM 505 and a non-volatile memory 506. The microprocessor 503, which may be, for example, a G3 or G4 microprocessor from Motorola, Inc. or IBM is coupled to cache memory 504 as shown in the example of FIG. 21. The bus 502 interconnects these various components together and also interconnects these components 503, 507, 505, and 506 to a display controller and display device 508 and to peripheral devices such as input/output (I/O) devices which may be mice, keyboards, modems, network interfaces, printers, scanners, video cameras and other devices which are well known in the art. Typically, the input/output devices 510 are coupled to the system through input/output controllers 509. The volatile RAM 505 is typically implemented as dynamic RAM (DRAM) which requires power continually in order to refresh or maintain the data in the memory. The non-volatile memory 506 is typically a magnetic hard drive or a magnetic optical drive or an optical drive or a DVD RAM or other type of memory systems which maintain data even after power is removed from the system. Typically, the non-volatile memory will also be a random access memory although this is not required. While FIG. 21 shows that the non-volatile memory is a local device coupled directly to the rest of the components in the data processing system, it will be appreciated that the present invention may utilize a non-volatile memory which is remote from the system, such as a network storage device which is coupled to the data processing system through a network interface such as a modem or Ethernet interface. The bus 502 may include one or more buses connected to each other through various bridges, controllers and/or adapters as is well known in the art. In one embodiment the I/O controller 509 includes a USB (Universal Serial Bus) adapter for controlling USB peripherals, and/or an EEE-1394 bus adapter for controlling IEEE-1394 peripherals.

In one embodiment of the present invention, ECG measurement system 511 (and/or measurement systems for other cardiac parameters, hemodynamic parameters, ventilation parameters, chest position/movement, position of operation platform relative to a reference position) is coupled to I/O controller 509 so that the data processing system 501 can gate the playback of pre-recorded images (e.g., stored on nonvolatile memory 506). Magnetic Position determination system 512 (or ultrasound or radio frequency based tracking system) is coupled to I/O controller 509 so that the data processing system determines the position relative to the heart in images played back according to the input from ECG measurement system. In one embodiment of the present invention, data processing system 501 performs the image processing based on stored image matrices to provide different views, image slices, surfaces and others according to real time condition. In one embodiment of the present invention, data processing system 501 is also used to perform data processing for the imaging system (e.g., a CT or MRI based imaging system). Alternatively, data processing system 501 receives image data through a communication link (e.g., network interface 510) or a removable medium (e.g., a zip diskette, a CD-R or DVD-R diskette, removable hard drive, and others).

It will be apparent from this description that aspects of the present invention may be embodied, at least in part, in software. That is, the techniques may be carried out in a computer system or other data processing system in response to its processor, such as a microprocessor, executing sequences of instructions contained in a memory, such as ROM 507, volatile RAM 505, non-volatile memory 506, cache 504 or a remote storage device. In various embodiments, hardwired circuitry may be used in combination with software instructions to implement the present invention. Thus, the techniques are not limited to any specific combination of hardware circuitry and software nor to any particular source for the instructions executed by the data processing system. In addition, throughout this description, various functions and operations are described as being performed by or caused by software code to simplify description. However, those skilled in the art will recognize what is meant by such expressions is that the functions result from execution of the code by a processor, such as the microprocessor 503.

A machine readable media can be used to store software and data which when executed by a data processing system causes the system to perform various methods of the present invention. This executable software and data may be stored in various places including for example ROM 507, volatile RAM 505, non-volatile memory 506 and/or cache 504 as shown in FIG. 21. Portions of this software and/or data may be stored in any one of these storage devices.

Thus, a machine readable media includes any mechanism that provides (i.e., stores and/or transmits) information in a form accessible by a machine (e.g., a computer, network device, personal digital assistant, manufacturing tool, any device with a set of one or more processors, etc.). For example, a machine readable media includes recordable/non-recordable media (e.g., read only memory (ROM); random access memory (RAM); magnetic disk storage media; optical storage media; flash memory devices; etc.), as well as electrical, optical, acoustical or other forms of propagated signals (e.g., carrier waves, infrared signals, digital signals, etc.); etc.

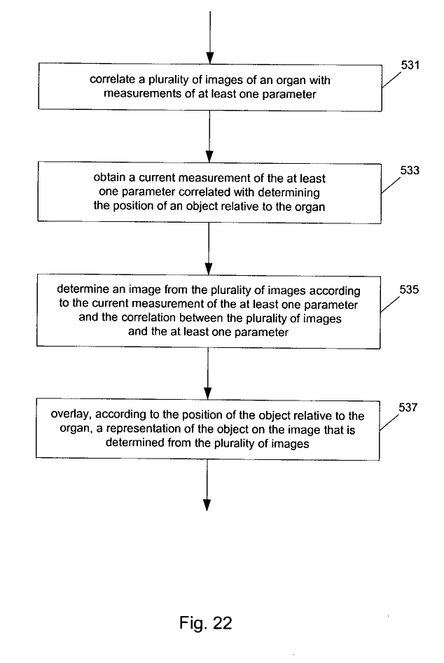

FIG. 22 shows a flow chart for a method to determine an image from a plurality of pre-recorded images to guide a portion of a device in real time by use of real time position tracking of a portion of that device during a percutaneous procedure according to one embodiment of the present invention. Operation 531 correlates a plurality of images of an organ (e.g., a heart) with measurements of at least one parameter (e.g., timing with respect to ECG signals). Operation 533 obtains a current measurement of the at least one parameter correlated with determining the position of an object (e.g., a catheter tip) relative to the organ. Operation 535 determines an image from the plurality of images according to the current measurement of the at least one parameter and the correlation between the plurality of images and the at least one parameter. Operation 537 overlays, according to the position of the object relative to the organ, a representation of the object on the image that is determined from the plurality of images to display the object in relation with the organ.

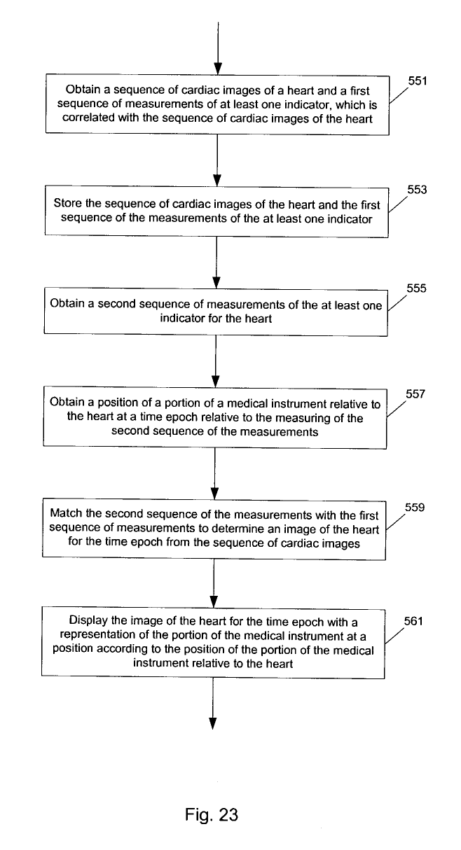

FIG. 23 shows a flow chart for a method of image guided real time device positioning using real time position tracking for a cardiac therapy according to one embodiment of the present invention. Operation 551 obtains a sequence of cardiac images of a heart and a first sequence of measurements of at least one indicator, which is correlated with the sequence of cardiac images of the heart. Operation 553 stores the sequence of cardiac images of the heart and the first sequence of the measurements of the at least one indicator. Operation 555 obtains a second sequence of measurements of the at least one indicator for the heart. Operation 557 obtains a position of a portion of a medical instrument relative to the heart at a time epoch relative to the measuring of the second sequence of the measurements. Operation 559 matches the second sequence of the measurements with the first sequence of measurements to determine an image of the heart for the time epoch from the sequence of cardiac images. Operation 561 displays the image of the heart for the time epoch with a representation of the portion of the medical instrument at a position according to the position of the portion of the medical instrument relative to the heart. In one embodiment of the present invention, the measurement of the second sequence is performed in real time to gate the playback of the sequence of the cardiac images in real time to show the state of the heart in real time. Further, the position of the portion of the medical instrument is determined in real time and superposed on the displayed image in real time to illustrate the position of the portion of the medical instrument in relation with the hard in real time.

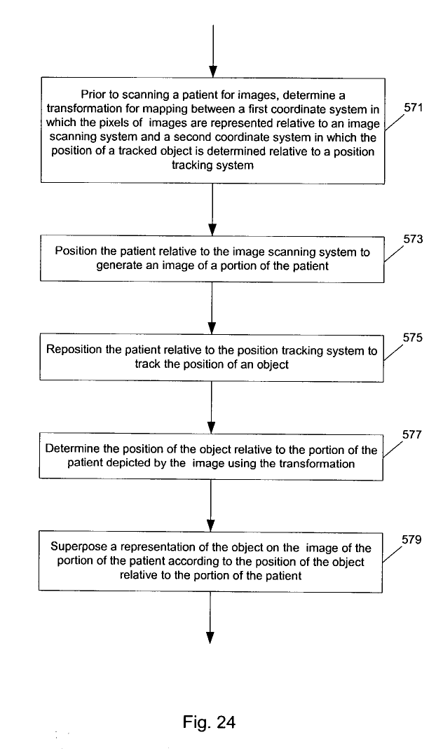

FIG. 24 shows a flow chart for a method to superpose a position determined by a position tracking system on an image from an imaging system according to one embodiment of the present invention. Prior to scanning a patient for images, operation 571 determines a transformation for mapping between a first coordinate system in which the pixels of images are represented relative to an image scanning system and a second coordinate system in which the position of a tracked object is determined relative to a position tracking system. The transformation specifies the geometrical relationship between the first and second coordinate systems such that the first and second coordinate systems can be aligned to overlain one over another with respect to a reference object, which is at a first reference position in the imaging system and at a second reference position in the position determination system. Operation 573 positions the patient relative to the image scanning system to generate an image of a portion of the patient. Operation 575 repositions the patient relative to the position tracking system to track the position of an object (e.g., tracking the tip of a catheter for cardiac therapy after the patient is transported from the imaging system to the Cath Lab). Operation 577 determines the position of the object relative to the portion of the patient depicted by the image using the transformation and the position information from the position tracking system. Operation 579 superposes a representation of the object on the image of the portion of the patient according to the position of the object relative to the portion of the patient.

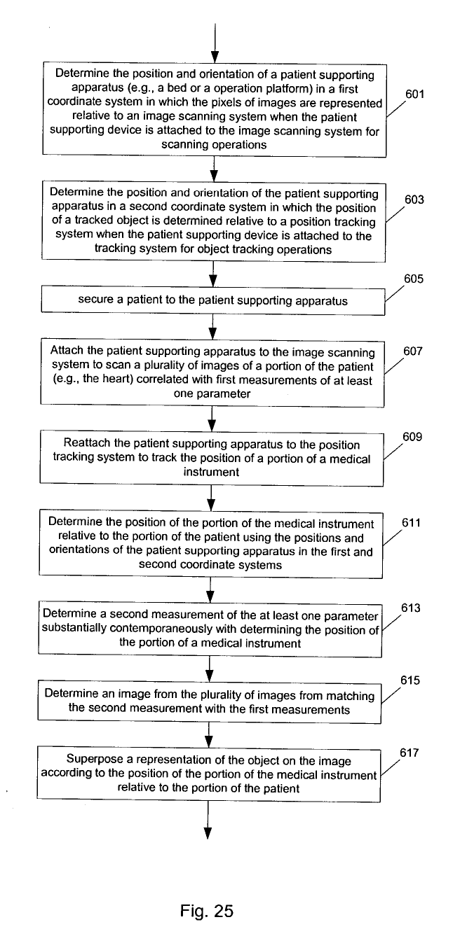

FIG. 25 shows a flow chart for a detailed method to superpose a position determined by a position tracking system on an image from an imaging system according to one embodiment of the present invention. Operation 601 determines the position and orientation of a patient supporting apparatus (e.g., a bed or a operation platform) in a first coordinate system in which the pixels of images are represented relative to an image scanning system when the patient supporting device is attached to the image scanning system for scanning operations. Operation 603 determines the position and orientation of the patient supporting apparatus in a second coordinate system in which the position of a tracked object is determined relative to a position tracking system when the patient supporting device is attached to the tracking system for object tracking operations. Operations 601 and 605 can be performed as an installation procedure in setting up the position tracking system and the imaging system, or as a calibration operation before the diagnosis and treatment of the patient, or a part of the diagnosis and treatment process.

Operation 605 secures a patient to the patient supporting apparatus. After operation 607 attaches the patient supporting apparatus to the image scanning system to scan a plurality of images of a portion of the patient (e.g., the heart) correlated with first measurements of at least one parameter, operation 609 reattaches the patient supporting apparatus to the position tracking system to track the position of a portion of a medical instrument. Operation 611 determines the position of the portion of the medical instrument relative to the portion of the patient using the positions and orientations of the patient supporting apparatus in the first and second coordinate systems. After operation 613 determines a second measurement of the at least one parameter substantially contemporaneously with determining the position of the portion of a medical instrument, operation 615 determines an image from the plurality of images from matching the second measurement with the first measurements. Operation 617 superposes a representation of the object on the image according to the position of the portion of the medical instrument relative to the portion of the patient.

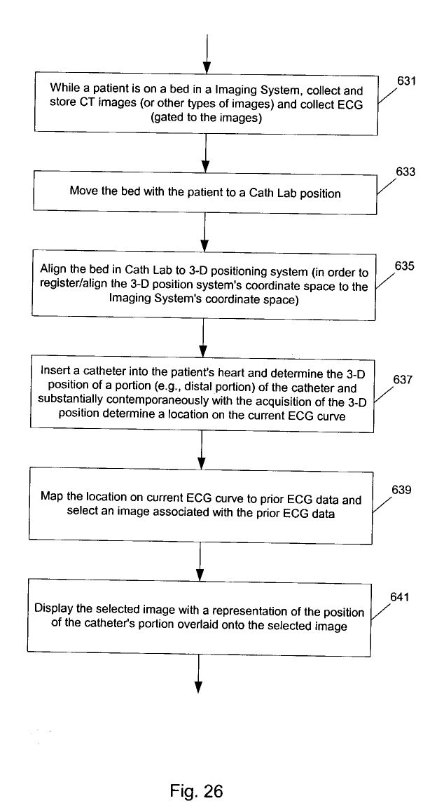

FIG. 26 shows a flow chart for a detailed method to guide a cardiac therapy using pre-recorded cardiac images according to one embodiment of the present invention. While a patient is on a bed in an Imaging System, operation 631 collects and stores CT images (or other types of images) and collects ECG (gated to the images). After operation 633 moves the bed with the patient to a Cath Lab position, operation 635 aligns the bed in Cath Lab to 3-D positioning system (in order to register/align the 3-D position system's coordinate space to the Imaging System's coordinate space). Operation 637 inserts a catheter into the patient's heart and determines the 3-D position of a portion (e.g., distal portion) of the catheter and substantially contemporaneously with the acquisition of the 3-D position determine a location on the current ECG curve. Operation 639 maps the location on current ECG curve to prior ECG data to select an image associated with the prior ECG data. Operation 641 displays the selected image with a representation of the position of the catheter's portion overlaid onto the selected image.

Although various embodiments are illustrated in the context of cardiac therapies, from this description, it will be apparent to one skilled in the art that similar approaches can also be applied to other percutaneous, for example, guiding an access/venogram catheter to the Coronary Sinus, guiding a pacing lead into the vein branch closest to the desired cardiac location, guiding an annuloplasty or other valve repair or replacement procedure, guiding and recording intra-cardiac injections and spatial dosing, guiding a device to a desired intra-cardiac diagnostic and/or anatomical location, guiding a device to a desired location within a coronary artery or vein, and others.

When the pre-recorded images are used to guide the operations, the use of conventional fluoroscopy during the operation can be avoided or minimized, along with the x-ray exposure risks for the attendant. In the procedures according to embodiments of the present invention, pre-recorded images are displayed according to the current measured parameters to guide the operation. In a conventional approach, an Interventional Cardiologist uses images from fluoroscopy to guide the operation.

When the present invention is used in an XMRI Cath Lab, the calibration operation to align the image coordinate space and the position tracking coordinate space can be automated and be relatively transparent to the physician/operator. As described above, the patient, the MRI and 3-D location equipments can be physically tied to one another in a known/controlled dimensional relationship so that the calibration functions can be performed using a phantom, performed as a part of regular equipment maintenance and/or simply be a part of the installation procedure. An XMRI Cath Lab will give the Interventional Cardiologist direct access and control of the 3-D MRI imaging of his patients and their hemodynamic state. Thus, this approach fits the normal Cath Lab patient processing procedures, potentially very complimentary to the XMRI systems adopted in the Cath Lab.

According to one embodiment of the present invention, a set of previously recorded (and, when desired, annotated/enhanced) ECG gated/timed 3-D image matrices (the diagnostic/anatomical map) produced by an x-ray and/or nuclear magnetic resonance system is used with a 3-D location system to streamline the therapeutic procedure. By overlaying the previously recorded 3-D diagnostic/anatomical maps in synchrony with the real time ECG with the real time catheter/device location, the system provides visual images to actually guide the therapy/device to the desired location(s). The locations of the previously applied therapy can also be record and overlaid on the diagnostic/anatomical map.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A method of displaying images of a heart, the method comprising:
storing a time-related sequence of cardiac images of a heart, the time-related sequence of cardiac images associated with at least one cardiac data parameter;
determining a position of a portion of a medical instrument relative to the heart;
determining at least one measurement of the at least one cardiac data parameter;
selecting at least one cardiac image from the time-related sequence of cardiac images according to the at least one measurement of the at least one cardiac data parameter; and
overlaying a representation of the position onto the at least one cardiac image to indicate the portion relative to the heart.

2. A method as in claim 1 wherein the at least one cardiac image is displayed to show the portion of the medical instrument in relation to the heart in real time; and, wherein the at least one measurement is determined substantially contemporaneously with the determining of the position.

3. A method as in claim 1 wherein the time-related sequence of cardiac images is correlated with measurements of the at least one cardiac data parameter;
wherein each of the time-related sequence of cardiac images comprises a pixel image; and, wherein the time-related sequence of cardiac images are generated from an imaging system based on at least one of:
a) Magnetic Resonance Imaging;
b) X-ray imaging; and
c) ultrasound imaging.

4. A method as in claim 1 wherein the at least one cardiac data parameter comprises at least one of:
a) Electrocardiogram (ECG);
b) heart sound;
c) blood pressure;
d) ventricular volume;
e) pulse wave;
f) heart motion; and
g) cardiac output.

5. A method as in claim 4 wherein said selecting is further based on a hemodynamic state determined substantially contemporaneously with the determining of the position; and, the hemodynamic state comprises at least one of:
a) blood pressure;
b) heart rate;
c) hydration state;
d) blood volume;
e) sedation state;
f) ventilation state; and
g) respiration state;
wherein the position is determined using a position determination system based on one of:
a) magnetic field;
b) ultrasound;
c) radio frequency signal; and
d) light.

6. A method as in claim 1 further comprising:
recording the position relative to the heart with annotation information; and
displaying a prior recorded position relative to the heart with annotation associated with the prior recorded position;
wherein the prior recorded position is overlaid onto the at least one cardiac image with the annotation associated with the prior recorded position.

7. A method as in claim 6 wherein the annotation information comprises at least one of:
a) an icon
b) a symbol;
c) a color coding;
d) entered writing;
e) a time;
f) data from a sensor;
g) data from a diagnostic device; and
h) data from a therapeutic device.

8. A method of displaying images to guide a medical operation, the method comprising:
determining a first state of an organ from at least one first measurement of at least one parameter; and
determining a first image from a plurality of images of the organ to display the organ in the first state, the plurality of images corresponding to the organ in a plurality of states.

9. A method as in claim 8 further comprising:
determining a first position of a portion of a medical instrument relative to the organ in the medical operation when the organ is in the first state.

10. A method as in claim 9 further comprising:
displaying the first image with a representation of the portion of the medical instrument overlaid on the first image according to the first position;
wherein the first image is displayed substantially in real time to show the portion of the medical instrument in relation with the organ.

11. A method as in claim 9 further comprising:
receiving data representing the at least one first measurement from at least one sensor; and
overlaying a representation of the portion of the medical instrument onto the first image to show the first position of the portion of the medical instrument in relation with the organ.

12. A method as in claim 9 wherein said determining the first position comprises:
receiving position information of the portion of the medical instrument from a position determination system when the organ is in the first state;
wherein the first position is determined from the position information from aligning both a first coordinate space of the position determination system and a second coordinate space of the plurality of images with respect to the organ;
wherein the first coordinate space and the second coordinate space are aligned with respect to the organ using a transformation to align the first coordinate space and the second coordinate space with respect to a reference object; and
wherein the reference object is a platform supporting a host of the organ; and,
wherein the host has a fixed position relative to the platform both when the plurality of images are generated in an imaging system and when the position information is determined in the position determination system.

13. A method as in claim 8 further comprising:
determining a second state of the organ from at least one second measurement of at least one parameter; and
determining a second image from the plurality of images of the organ to display the organ in the second state;
wherein the first and second images are determined substantially in real time to show the organ in the first and second states;
wherein the plurality of images is obtained prior to the medical operation;
wherein the first and second images are displayed to guide the medical operation; and
wherein the first and second images are automatically determined according to the at least one parameter in real time during the medical operation.

14. A method of displaying images to guide a medical operation, the method comprising:
storing a plurality of images of an organ, the plurality of images associated with at least one parameter; and
automatically playing back the plurality of images in real time according to real time measurements of the at least one parameter.

15. A method as in claim 14 further comprising:
receiving position information of a portion of a medical instrument in real time during the medical operation;
overlaying a representation of the portion of the medical instrument on displayed ones of the plurality of images to illustrate a position of the portion of the medical instrument in relation with the organ according to the position information; and
determining a position of the portion of the medical instrument relative to the organ in a displayed one of the plurality of images from the position information;
wherein the position information is determined by a real time position tracking system based on one of:
a) magnetic field;
b) ultrasound;
c) radio frequency signal; and
d) light.

16. A method as in claim 14 wherein the plurality of images are obtained before said playing back; wherein the plurality of images are obtained using a Magnetic Resonance Imaging (MRI) system; and, wherein the plurality of images are obtained using a Computer Tomography (CT) system.

17. A method to display an image for guiding a medical operation, the method comprising:
collecting an image of an organ of a person, the image being generated by an imaging system and in a coordinate system of the imaging system while the person is in a first position relative to a platform in the imaging system;
collecting first position information that represents a position of a portion of a medical instrument in a coordinate system of a position determination system, the first position information being generated by the position determination system while the person is in the first position relative to the platform in the position determination system after the person and the platform are transported from the imaging system to the position determination system;
determining a second position that is a position of the portion of the medical instrument relative to the organ depicted in the image and is derived from the first position information; and
overlaying a representation of the portion of the medical instrument onto the image of the organ according to the second position to display the position of the portion of the medical instrument relative to the organ.

18. A method as in claim 17 further comprising:
transporting the person with the platform from the imaging system to the position determination system while the person remains in the first position relative to the platform.

19. A method as in claim 17 wherein the second position is determined using predetermined data that relates the coordinate system of the position determination system and the coordinate system of the imaging system.

20. A method as in claim 19 wherein the predetermined data specifies a transformation to align a position of the platform, which is generated by the position determination system when the platform is in a third position that is in the position determination system, with a corresponding position of the platform on an image, which is generated by the imaging system when the platform is in a fourth position that is in the imaging system; and, wherein the predetermined data is comprises data representing a position and orientation of the platform in the coordinate system of the position determination system when the platform is in the third position.

21. A method as in claim 20 wherein the predetermined data further comprises data representing a position and orientation of the platform in the coordinate system of the imaging system when the platform is in the fourth position.

22. A method as in claim 21 wherein the image of the organ is collected when the platform is in the fourth position; and, wherein the first position information is collected when the platform is in the third position.

23. A method as in claim 21 further comprising:
receiving second position information, the second position information indicating a position of the platform relative to the third position when the first position information is collected.

24. A method as in claim 21 further comprising:
receiving third position information, the third position information indicates a position of the platform relative to the fourth position when the image of the organ is collected.

25. A method as in claim 19 wherein the predetermined data is determined before the image of the organ is generated; and, wherein the predetermined data is determined without the person.

26. A method to determine a position of a portion of a medical instrument relative to an organ, the method comprising:
receiving data for aligning overlaying positions determined by a position determination system relative to a reference object with corresponding positions on images generated from an imaging system relative to the reference object, the reference object being at a first position in the imaging system when the images are generated, the reference object being at a second position in the position determination system when the positions are determined;
receiving position information of the portion of the medical instrument determined by the position determination system, the position information being determined when the reference object is in a third position relative to the organ in the position determination system;
determining a position of the portion of the medical instrument relative to the organ depicted in a first image from the position information and the data, the first image being generated by the imaging system when the reference object is in the third position relative to the organ in the imaging system.

27. A method as in claim 26 wherein the data comprises.
a) data representing a position of the reference object determined by the position determination system when the reference object is in the second position;
b) data representing an orientation of the reference object determined by the position determination system when the reference object is in the second position;
c) data representing a position of the reference object in an image generated from the imaging system when the reference object is in the first position; and
d) data representing an orientation of the reference object in an image generated from the imaging system when the reference object is in the first position.

28. A method as in claim 27 wherein the position of the portion of the medical instrument relative to the organ is determined using one of:
a) data indicating a position of the reference object relative to the second position when the position information is determined; and
b) data indicating a position of the reference object relative to the first position when the first image is generated.

29. A method as in claim 26 wherein the reference object is a platform for supporting a host of the organ; and, wherein the organ is a heart.

30. A method as in claim 26 wherein the first image is selected from a plurality of images of the organ according to at least one measurement of at least one parameter related to the organ, the at least one measurement generated substantially contemporaneous with a time at which the position information is determined, the plurality of images associated with different measurements of the at least one parameter.

31. A machine readable medium containing executable computer program instructions which when executed by a data processing system cause said system to perform a method of displaying images of a heart, the method comprising:
storing a time-related sequence of cardiac images of a heart, the time-related sequence of cardiac images associated with at least one cardiac data parameter;
determining a position of a portion of a medical instrument relative to the heart;
determining at least one measurement of the at least one cardiac data parameter;
selecting at least one cardiac image from the time-related sequence of cardiac images according to the at least one measurement of the at least one cardiac data parameter; and
overlaying a representation of the position onto the at least one cardiac image to indicate the portion relative to the heart.

32. A medium as in claim 31 wherein the at least one cardiac image is displayed to show the portion of the medical instrument in relation to the heart in real time; and, wherein the at least one measurement is determined substantially contemporaneously with the determining of the position.

33. A medium as in claim 31 wherein the time-related sequence of cardiac images is correlated with measurements of the at least one cardiac data parameter; wherein each of the time-related sequence of cardiac images comprises a pixel image; and, wherein the time-related sequence of cardiac images are generated from an imaging system based on at least one of:
a) Magnetic Resonance Imaging;
b) X-ray imaging; and
c) ultrasound imaging.

34. A medium as in claim 31 wherein the at least one cardiac data parameter comprises at least one of:
a) Electrocardiogram (ECG);
b) heart sound;
c) blood pressure;
d) ventricular volume;
e) pulse wave;
f) heart motion; and
g) cardiac output.

35. A medium as in claim 34 wherein said selecting is further based on a hemodynamic state determined substantially contemporaneously with the determining of the position; and, wherein the hemodynamic state comprises at least one of:
a) blood pressure;
b) heart rate;
c) hydration state;
d) blood volume;
e) sedation state;
f) ventilation state; and
g) respiration state;
wherein the position is determined using a position determination system based on one of:
a) magnetic field;
b) ultrasound;
c) radio frequency signal; and
d) light.

36. A medium as in claim 31 wherein the method further comprises:
recording the position relative to the heart with annotation information; and
displaying a prior recorded position relative to the heart with annotation associated with the prior recorded position;
wherein the prior recorded position is overlaid onto the at least one cardiac image with the annotation associated with the prior recorded position.

37. A medium as in claim 36 wherein the annotation information comprises at least one of:
a) an icon
b) a symbol;
c) a color coding;
d) entered writing;
e) a time;
f) data from a sensor;
g) data from a diagnostic device; and
h) data from a therapeutic device.

38. A machine readable medium containing executable computer program instructions which when executed by a data processing system cause said system to perform a method of displaying images to guide a medical operation, the method comprising:
determining a first state of an organ from at least one first measurement of at least one parameter; and
determining a first image from a plurality of images of the organ to display the organ in the first state, the plurality of images corresponding to the organ in a plurality of states.

39. A medium as in claim 38 wherein the method further comprises:
determining a first position of a portion of a medical instrument relative to the organ in the medical operation when the organ is in the first state.

40. A medium as in claim 39 wherein the method further comprises:
displaying the first image with a representation of the portion of the medical instrument overlaid on the first image according to the first position;
wherein the first image is displayed substantially in real time to show the portion of the medical instrument in relation with the organ.

41. A medium as in claim 39 wherein the method further comprises:
receiving data representing the at least one first measurement from at least one sensor; and
overlaying a representation of the portion of the medical instrument onto the first image to show the first position of the portion of the medical instrument in relation with the organ.

42. A medium as in claim 39 wherein said determining the first position comprises:
receiving position information of the portion of the medical instrument from a position determination system when the organ is in the first state;
wherein the first position is determined from the position information from aligning both a first coordinate space of the position determination system and a second coordinate space of the plurality of images with respect to the organ;
wherein the first coordinate space and the second coordinate space are aligned with respect to the organ using a transformation to align the first coordinate space and the second coordinate space with respect to a reference object;
wherein the reference object is a platform supporting a host of the organ; and,
wherein the host has a fixed position relative to the platform both when the plurality of images are generated in an imaging system and when the position information is determined in the position determination system.

43. A medium as in claim 38 wherein the method further comprises:
determining a second state of the organ from at least one second measurement of at least one parameter; and
determining a second image from the plurality of images of the organ to display the organ in the second state;
wherein the first and second images are determined substantially in real time to show the organ in the first and second states;
wherein the plurality of images is obtained prior to the medical operation;
wherein the first and second images are displayed to guide the medical operation; and
wherein the first and second images are automatically determined according to the at least one parameter in real time during the medical operation.

44. A machine readable medium containing executable computer program instructions which when executed by a data processing system cause said system to perform a method of displaying images to guide a medical operation, the method comprising:
storing a plurality of images of an organ, the plurality of images associated with at least one parameter; and
automatically playing back the plurality of images in real time according to real time measurements of the at least one parameter.

45. A medium as in claim 44 wherein the method further comprises:
receiving position information of a portion of a medical instrument in real time during the medical operation;
overlaying a representation of the portion of the medical instrument on displayed ones of the plurality of images to illustrate a position of the portion of the medical instrument in relation with the organ according to the position information; and
determining a position of the portion of the medical instrument relative to the organ in a displayed one of the plurality of images from the position information;
wherein the position information is determined by a real time position tracking system based on one of:
a) magnetic field;
b) ultrasound;
c) radio frequency signal; and
d) light.

46. A medium as in claim 44 wherein the plurality of images are obtained before said playing back; wherein the plurality of images are obtained using a Magnetic Resonance Imaging (MRI) system; and, wherein the plurality of images are obtained using a Computer Tomography (CT) system.

47. A machine readable medium containing executable computer program instructions which when executed by a data processing system cause said system to perform a method to display an image for guiding a medical operation, the method comprising:
collecting an image of an organ of a person, the image being generated by an imaging system and in a coordinate system of the imaging system while the person is in a first position relative to a platform in the imaging system;
collecting first position information that represents a position of a portion of a medical instrument in a coordinate system of a position determination system, the first position information being generated by the position determination system while the person is in the first position relative to the platform in the position determination system after the person and the platform are transported from the imaging system to the position determination system;
determining a second position that is a position of the portion of the medical instrument relative to the organ depicted in the image and is derived from the first position information; and
overlaying a representation of the portion of the medical instrument onto the image of the organ according to the second position to display the position of the portion of the medical instrument relative to the organ.

48. A medium as in claim 47 wherein the second position is determined using predetermined data that relates the coordinate system of the position determination system and the coordinate system of the imaging system.

49. A medium as in claim 48 wherein the predetermined data specifies a transformation to align a position of the platform, which is generated by the position determination system when the platform is in a third position that is in the position determination system, with a corresponding position of the platform on an image, which is generated by the imaging system when the platform is in a fourth position that is in the imaging system; the predetermined data is comprises data representing a position and orientation of the platform in the coordinate system of the position determination system when the platform is in the third position.

50. A medium as in claim 49 wherein the predetermined data further comprises data representing a position and orientation of the platform in the coordinate system of the imaging system when the platform is in the fourth position.

51. A medium as in claim 50 wherein the image of the organ is collected when the platform is in the fourth position; and, wherein the first position information is collected when the platform is in the third position.

52. A medium as in claim 50 wherein the method further comprises:
receiving second position information, the second position information indicating a position of the platform relative to the third position when the first position information is collected.

53. A medium as in claim 50 wherein the method further comprises:
receiving third position information, the third position information indicates a position of the platform relative to the fourth position when the image of the organ is collected.

54. A medium as in claim 48 wherein the predetermined data is determined before the image of the organ is generated; and, wherein the predetermined data is determined without the person.

55. A machine readable medium containing executable computer program instructions which when executed by a data processing system cause said system to perform a method to determine a position of a portion of a medical instrument relative to an organ, the method comprising:
receiving data for aligning overlaying positions determined by a position determination system relative to a reference object with corresponding positions on images generated from an imaging system relative to the reference object, the reference object being at a first position in the imaging system when the images are generated, the reference object being at a second position in the position determination system when the positions are determined;
receiving position information of the portion of the medical instrument determined by the position determination system, the position information being determined when the reference object is in a third position relative to the organ in the position determination system;
determining a position of the portion of the medical instrument relative to the organ depicted in a first image from the position information and the data, the first image being generated by the imaging system when the reference object is in the third position relative to the organ in the imaging system.

56. A medium as in claim 55 wherein the data comprises:
a) data representing a position of the reference object determined by the position determination system when the reference object is in the second position;
b) data representing an orientation of the reference object determined by the position determination system when the reference object is in the second position;
c) data representing a position of the reference object in an image generated from the imaging system when the reference object is in the first position; and
d) data representing an orientation of the reference object in an image generated from the imaging system when the reference object is in the first position.

57. A medium as in claim 56 wherein the position of the portion of the medical instrument relative to the organ is determined using one of:
a) data indicating a position of the reference object relative to the second position when the position information is determined; and
b) data indicating a position of the reference object relative to the first position when the first image is generated.

58. A medium as in claim 55 wherein the reference object is a platform for supporting a host of the organ; and, wherein the organ is a heart.

59. A medium as in claim 55 wherein the first image is selected from a plurality of images of the organ according to at least one measurement of at least one parameter related to the organ, the at least one measurement generated substantially contemporaneous with a time at which the position information is determined, the plurality of images associated with different measurements of the at least one parameter.

60. A data processing system to display images of a heart, the data processing system comprising:
means for storing a time-related sequence of cardiac images of a heart, the time-related sequence of cardiac images associated with at least one cardiac data parameter;
means for determining a position of a portion of a medical instrument relative to the heart;
means for determining at least one measurement of the at least one cardiac data parameter;
means for selecting at least one cardiac image from the time-related sequence of cardiac images according to the at least one measurement of the at least one cardiac data parameter; and
means for overlaying a representation of the position onto the at least one cardiac image to indicate the portion relative to the heart.

61. A data processing system as in claim 60 wherein the at least one cardiac image is displayed to show the portion of the medical instrument in relation to the heart in real time; and, wherein the at least one measurement is determined substantially contemporaneously with the determining of the position.

62. A data processing system as in claim 60 wherein the time-related sequence of cardiac images is correlated with measurements of the at least one cardiac data parameter; wherein each of the time-related sequence of cardiac images comprises a pixel image; and, wherein the time-related sequence of cardiac images are generated from an imaging system based on at least one of:
a) Magnetic Resonance Imaging;
b) X-ray imaging; and
c) ultrasound imaging.

63. A data processing system as in claim 60 wherein the at least one cardiac data parameter comprises at least one of:
a) Electrocardiogram (ECG);
b) heart sound;
c) blood pressure;
d) ventricular volume;
e) pulse wave;
f) heart motion; and
g) cardiac output.

64. A data processing system as in claim 63 wherein the at least one cardiac image is selected based on a hemodynamic state determined substantially contemporaneously with the determining of the position; and, wherein the hemodynamic state comprises at least one of:
a) blood pressure;
b) heart rate;
c) hydration state;
d) blood volume;
e) sedation state;
f) ventilation state; and
g) respiration state;
wherein the position is determined using a position determination system based on one of:
a) magnetic field;
b) ultrasound;
c) radio frequency signal; and
d) light.

65. A data processing system as in claim 60 further comprising:
means for recording the position relative to the heart with annotation information; and
means for displaying a prior recorded position relative to the heart with annotation associated with the prior recorded position;
wherein the prior recorded position is overlaid onto the at least one cardiac image with the annotation associated with the prior recorded position.

66. A data processing system as in claim 65 wherein the annotation information comprises at least one of:
a) an icon
b) a symbol;
c) a color coding;
d) entered writing;
e) a time;
f) data from a sensor;
g) data from a diagnostic device; and
h) data from a therapeutic device.

67. A data processing system to display images to guide a medical operation, the data processing system comprising:
means for determining a first state of an organ from at least one first measurement of at least one parameter; and
means for determining a first image from a plurality of images of the organ to display the organ in the first state, the plurality of images corresponding to the organ in a plurality of states.

68. A data processing system as in claim 67 further comprising:
means for determining a first position of a portion of a medical instrument relative to the organ in the medical operation when the organ is in the first state.

69. A data processing system as in claim 68 further comprising:
means for displaying the first image with a representation of the portion of the medical instrument overlaid on the first image according to the first position;
wherein the first image is displayed substantially in real time to show the portion of the medical instrument in relation with the organ.

70. A data processing system as in claim 68 further comprising:
means for receiving data representing the at least one first measurement from at least one sensor; and
means for overlaying a representation of the portion of the medical instrument onto the first image to show the first position of the portion of the medical instrument in relation with the organ.

71. A data processing system as in claim 68 wherein said means for determining the first position comprises:
means for receiving position information of the portion of the medical instrument from a position determination system when the organ is in the first state;
wherein the first position is determined from the position information from aligning both a first coordinate space of the position determination system and a second coordinate space of the plurality of images with respect to the organ;
wherein the first coordinate space and the second coordinate space are aligned with respect to the organ using a transformation to align the first coordinate space and the second coordinate space with respect to a reference object; and
wherein the reference object is a platform supporting a host of the organ; and,
wherein the host has a fixed position relative to the platform both when the plurality of images are generated in an imaging system and when the position information is determined in the position determination system.

72. A data processing system as in claim 67 further comprising:
means for determining a second state of the organ from at least one second measurement of at least one parameter; and
means for determining a second image from the plurality of images of the organ to display the organ in the second state;
wherein the first and second images are determined substantially in real time to show the organ in the first and second states;
wherein the plurality of images is obtained prior to the medical operation;
wherein the first and second images are displayed to guide the medical operation; and
wherein the first and second images are automatically determined according to the at least one parameter in real time during the medical operation.

73. A data processing system to display images to guide a medical operation, the data processing system comprising:
means for storing a plurality of images of an organ, the plurality of images associated with at least one parameter; and
means for automatically playing back the plurality of images in real time according to real time measurements of the at least one parameter.

74. A data processing system as in claim 73 further comprising:
means for receiving position information of a portion of a medical instrument in real time during the medical operation
means for overlaying a representation of the portion of the medical instrument on displayed ones of the plurality of images to illustrate a position of the portion of the medical instrument in relation with the organ according to the position information; and
means for determining a position of the portion of the medical instrument relative to the organ in a displayed one of the plurality of images from the position information;
wherein the position information is determined by a real time position tracking system based on one of:
a) magnetic field;
b) ultrasound;
c) radio frequency signal; and
d) light.

75. A data processing system as in claim 73 wherein the plurality of images are obtained before the plurality of images is played back in real time; wherein the plurality of images are obtained using a Magnetic Resonance Imaging (MRI) system; and, wherein the plurality of images are obtained using a Computer Tomography (CT) system.

76. A data processing system to display an image for guiding a medical operation, the data processing system comprising:
means for collecting an image of an organ of a person, the image being generated by an imaging system and in a coordinate system of the imaging system while the person is in a first position relative to a platform in the imaging system;
means for collecting first position information that represents a position of a portion of a medical instrument in a coordinate system of a position determination system, the first position information being generated by the position determination system while the person is in the first position relative to the platform in the position determination system after the person and the platform are transported from the imaging system to the position determination system;
means for determining a second position that is a position of the portion of the medical instrument relative to the organ depicted in the image and is derived from the first position information; and
means for overlaying a representation of the portion of the medical instrument onto the image of the organ according to the second position to display the position of the portion of the medical instrument relative to the organ.

77. A data processing system as in claim 76 wherein the second position is determined using predetermined data that relates the coordinate system of the position determination system and the coordinate system of the imaging system.

78. A data processing system as in claim 77 wherein the predetermined data specifies a transformation to align a position of the platform, which is generated by the position determination system when the platform is in a third position that is in the position determination system, with a corresponding position of the platform on an image, which is generated by the imaging system when the platform is in a fourth position that is in the imaging system; and, wherein the predetermined data is comprises data representing a position and orientation of the platform in the coordinate system of the position determination system when the platform is in the third position.

79. A data processing system as in claim 78 wherein the predetermined data further comprises data representing a position and orientation of the platform in the coordinate system of the imaging system when the platform is in the fourth position.

80. A data processing system as in claim 79 wherein the image of the organ is collected when the platform is in the fourth position; and, wherein the first position information is collected when the platform is in the third position.

81. A data processing system as in claim 79 further comprising:
means for receiving second position information, the second position information indicating a position of the platform relative to the third position when the first position information is collected.

82. A data processing system as in claim 79 further comprising:
means for receiving third position information, the third position information indicates a position of the platform relative to the fourth position when the image of the organ is collected.

83. A data processing system as in claim 77 wherein the predetermined data is determined before the image of the organ is generated; and, wherein the predetermined data is determined without the person.

84. A data processing system to determine a position of a portion of a medical instrument relative to an organ, the data processing system comprising:
means for receiving data for aligning overlaying positions determined by a position determination system relative to a reference object with corresponding positions on images generated from an imaging system relative to the reference object, the reference object being at a first position in the imaging system when the images are generated, the reference object being at a second position in the position determination system when the positions are determined;
means for receiving position information of the portion of the medical instrument determined by the position determination system, the position information being determined when the reference object is in a third position relative to the organ in the position determination system;
means for determining a position of the portion of the medical instrument relative to the organ depicted in a first image from the position information and the data, the first image being generated by the imaging system when the reference object is in the third position relative to the organ in the imaging system.

85. A data processing system as in claim 84 wherein the data comprises:
a) data representing a position of the reference object determined by the position determination system when the reference object is in the second position;
b) data representing an orientation of the reference object determined by the position determination system when the reference object is in the second position;
c) data representing a position of the reference object in an image generated from the imaging system when the reference object is in the first position; and
d) data representing an orientation of the reference object in an image generated from the imaging system when the reference object is in the first position.

86. A data processing system as in claim 85 wherein the position of the portion of the medical instrument relative to the organ is determined using one of:
a) data indicating a position of the reference object relative to the second position when the position information is determined; and
b) data indicating a position of the reference object relative to the first position when the first image is generated.

87. A data processing system as in claim 84 wherein the reference object is a platform for supporting a host of the organ; and, wherein the organ is a heart.

88. A data processing system as in claim 84 wherein the first image is selected from a plurality of images of the organ according to at least one measurement of at least one parameter related to the organ, the at least one measurement generated substantially contemporaneous with a time at which the position information is determined, the plurality of images associated with different measurements of the at least one parameter.

89. A guiding system to guide a percutaneous procedure, the system comprising:
a data processing system, the data processing system comprising:
memory; and
a processor coupled to the memory;
an imaging system coupled to the data processing system, the image system generating a plurality of images of an organ, the plurality of images corresponding to the organ in a plurality of states, the memory storing the plurality of images, the data processing system receiving at least one measurement of at least one parameter, the processor determining a first state of the organ from the at least one measurement, the processor determining a first image from a plurality of images to display the organ in the first state.

90. A guiding system as in claim 89 further comprising:
a position determination system coupled to the data processing system, the position determination system determining first position information of a portion of a medical instrument when the organ is in the first state, the processor determining a first position of the portion of the medical instrument relative to the organ to display the first image with a representation of the portion of the medical instrument overlaid on the first image according to the first position.

91. A guiding system as in claim 90 wherein the first image is displayed substantially in real time to show the portion of the medical instrument in relation with the organ; wherein the plurality of images are played back in real time according to real time measurements of the at least one parameter to show states of the organ in real time; and, wherein the processor overlays a representation of the portion of the medical instrument in real time according to real time position information of the portion of the medical instrument obtained from the position determination system to illustrate the portion of the medical instrument in relation with the organ.

92. A guiding system as in claim 89 wherein the plurality of images are played back in real time according to real time measurements of the at least one parameter to show states of the organ in real time; and, wherein the plurality of images are generated before the plurality of images are played back in real time; and, wherein the position determination system uses sensors based on one of:
a) magnetic field;
b) ultrasound;
c) radio frequency signal; and
d) light;
wherein the imaging system is based on one of: Magnetic Resonance Imaging (MRI); and,
Computer Tomography (CT).

93. A guiding system as in claim 89 wherein the organ is a heart; and, wherein the plurality of images is correlated with measurements of the at least one parameter.

94. A guiding system as in claim 93 wherein the imaging system is based on at least one of:
a) Magnetic Resonance;
b) X-ray; and
c) ultrasound.
wherein the at least one parameter comprises at least one of:
a) Electrocardiogram (ECG);
b) heart sound;
c) blood pressure;
d) ventricular volume;
e) pulse wave;
f) heart motion; and
g) cardiac output.

95. A guiding system as in claim 90 wherein the first position is determined from aligning a first coordinate space of the position determination system and a second coordinate space of the imaging system with respect to the organ;
wherein the first coordinate space and the second coordinate space are aligned with respect to the organ using a transformation to align the first coordinate space and the second coordinate space with respect to a reference object; wherein the reference object is a platform supporting a host of the organ; wherein the host has a fixed position relative to the platform both when the plurality of images are generated in an imaging system and when the position information is determined in the position determination system.

96. A guiding system as in claim 95 further comprising:
a rail system coupled between the position determination system and the imaging system, the platform being transported between the imaging system and position determination system on the rail system.

97. A guiding system as in claim 90 further comprising:
a rail system coupled between the position determination system and the imaging system, the rail system supporting a platform in transporting the platform from the imaging system and position determination system, a host of the organ being in a fixed position relative to the platform when the plurality of images are generated and the first position information is determined;
wherein the first position is determined using data for overlaying positions determined by the position determination system onto a second image generated from the imaging system relative to a platform for supporting a host of the organ, the platform being at a second position in the position determination system when the positions are determined, the platform being at a third position in the imaging system when the second image is generated.

98. A guiding system as in claim 97 wherein the data comprises:
a) data representing a position and orientation of the platform determined by the position determination system when the platform is in the second position; and
b) data representing a position and orientation of the platform in an image generated from the imaging system when the platform is in the third position;
wherein the first position is determined using one of:
a) data indicating a position of the platform relative to the second position when the first position information is determined; and
b) data indicating a position of the platform relative to the third position when the first image is generated.
